SEQUENCE LISTING (16 sequences)

SEQ ID NO: 1 - protein, 451 aa, Mus musculus
EVQLQESGPSLVKPSQTLSLTCYVTGDSITSGYWNWIRKFPGNKLEYMGYINYSGSTYYNPSLKSRISIIRDTSQNHYNLLLNSVTTEDTATYYCARLGTYYRYAGAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 2 - protein, 219 aa, Mus musculus
DDVLTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 5 aa, Mus musculus
SGYWN

SEQ ID NO: 4 - protein, 16 aa, Mus musculus
YINYSGSTYYNPSLKS

SEQ ID NO: 5 - protein, 13 aa, Mus musculus
LGTYYRYAGAMDY

SEQ ID NO: 6 - protein, 16 aa, Mus musculus
RSSQSLVHSNGNTYLH

SEQ ID NO: 7 - protein, 7 aa, Mus musculus
KVSNRFS

SEQ ID NO: 8 - protein, 9 aa, Mus musculus
SQSTHVPYT

SEQ ID NO: 9 - DNA, 1410 bp, Mus musculus
atgatggtgttaagtcttctgtacctgttgacagcccttccgggtgtcctgtcagaagtacagcttcaggagtcaggacctagcctcgtgaaaccttctcagactctgtccctcacctgttatgtcactggcgactccatcaccagtggttactggaactggatccggaaattcccagggaataagcttgagtacatgggatacataaactacagtggttctacttactacaatccatctctcaaaagtcgaatctccatcattcgagacacatcccagaaccactacaacctgctgttgaattctgtgactactgaggacacagccacatattactgtgcaagattggggacctactataggtacgctggagctatggactactggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 10 - DNA, 717 bp, Mus musculus
atgaagttgcctgttaggctgttggtgctgctgttctggattcctgcttccagcagtgatgatgtgttgacccaaactccactctccctgcctgtcagtcttggagatcaagcctccatatcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 11 - DNA, 15 bp, Mus musculus
agtggttactggaac

SEQ ID NO: 12 - DNA, 48 bp, Mus musculus
tacataaactacagtggttctacttactacaatccatctctcaaaagt

SEQ ID NO: 13 - DNA, 39 bp, Mus musculus
ttggggacctactataggtacgctggagctatggactac

SEQ ID NO: 14 - DNA, 48 bp, Mus musculus
agatctagtcagagccttgtacacagtaatggaaacacctatttacat

SEQ ID NO: 15 - DNA, 21 bp, Mus musculus
aaagtttccaaccgattttct

SEQ ID NO: 16 - DNA, 27 bp, Mus musculus
tctcaaagtacacatgttccgtacacg

(19) United States
(12) Patent Application Publication
(10) Pub. No.: US 2021/0246223 A1
(43) Pub. Date: Aug. 12, 2021

(54) ANTI-HER3 Antibody and uses thereof

(71) Applicant: CRD PHARMACEUTICALS INC, Ottawa (CA)

(72) Inventor: Abedelnasser ABULROB, Ottawa (CA)

(21) Appl. No.: 16/973,400

(22) PCT Filed: Jun. 21, 2019
(86) PCT No.: PCT/CA2019/050869

§ 371 Date: Dec. 8, 2020

Related U.S. Application Data
(60) Provisional application No. 62/688,628, filed on Jun. 22, 2018.

(51) Int. Cl. C07K 16/32; A61K 45/06; A61K 39/395; A61P 35/00

(52) U.S. Cl. CPC C07K 16/32; A61K 45/06; A61K 39/39558; A61K 2039/505; C07K 2317/92; C07K 2317/33; A61P 35/00

(57) ABSTRACT

The present invention relates to antibodies that specifically bind human human epidermal growth factor receptor 3 (also known as ERBB3 or HER3), methods for their production, pharmaceutical compositions containing said antibodies, and uses thereof. The present invention also provides the antigen binding protein, the nucleic acid, the vector, the cell, or the pharmaceutical for use as a medicament. The present invention further provides a method of inhibiting tumor growth or treating cancer, comprising administering a therapeutically effective amount of the antigen binding protein, the fusion protein or conjugate, the nucleic acid, the vector, the cell, or the pharmaceutical.

### CROSS-REFERENCE TO RELATED APPLICATIONS

This application claims priority to, and the benefit of, U.S. Provisional Application No. 62/688,628, filed Jun. 22, 2018, the contents of which are incorporated herein by reference in their entirety.

### REFERENCE TO THE SEQUENCE LISTING

The present application is being filed along with a Sequence Listing in electronic format via EFS-Web. The Sequence Listing is provided as a text file entitled “Sequence listing”, which is 11 kilobyte in size. The information in the electronic format of the Sequence Listing is incorporated herein by reference in its entirety.

### BACKGROUND

### Field of the Invention

The present invention relates to antibodies that specifically bind human human epidermal growth factor receptor 3 (also known as ERBB3 or HER3 antibody), methods for their production, pharmaceutical compositions containing said antibodies, and uses thereof.

### Description of the Related Art

### SUMMARY

The human epidermal growth factor receptor 3 (ErbB3, also known as HER3) is a receptor protein tyrosine kinase and belongs to the epidermal growth factor receptor (EGFR) subfamily of receptor protein tyrosine kinases, which also includes EGFR (HER1, ErbBI), HER2 (ErbB2, Neu), and HER4 (ErbB4) (Plowman et al, (1990) Proc. Natl. Acad. Sci. U.S.A. 87:4905-4909; Kraus et al, (1989) PNAS 86:9193-9197; and Kraus et al, (1993) PNAS 90:2900-2904). Like the prototypical EGFR, the transmembrane receptor HER3 consists of an extracellular ligand-binding domain (ECD), a dimerization domain within the ECD, a transmembrane domain, an intracellular protein tyrosine kinase-like domain and a C-terminal phosphorylation domain. The ectodomains of the ErbB receptors are further characterized as being divided into four domains (I-IV). Domains I and III of the ErbB ectodomain are involved in ligand binding (see, e.g., Hynes et. al. (2005) Nature Rev. Cancer 5, 341-354). Unlike the other HER family members, the kinase domain of HER3 displays very low intrinsic kinase activity.

The complex signaling network of the ErbB family members is tightly regulated in normal human tissue. However, dysregulation of ErbB family members by receptor overexpression, alteration of receptor functions by mutations or aberrant stimulation by ligands is often associated with the development and propagation of cancer. EGFR is frequently overexpressed in colorectal cancer, ovarian cancer, head and neck squamous cell carcinoma and other cancer types and EGFR overexpression has been linked to poor prognosis. HER2 is particularly associated with human breast cancer, where it is amplified and/or overexpressed in up to 30%.

HER3 has potent activation of the PI3K/Akt pathway which has been reported to be responsible for resistance mechanisms against ErbB targeted therapies (Holbro et al., 2003, PNAS 100:8933-8938). For example, the overexpression of HER3 receptor is a marker of acquired resistance of lung cancer to gefitinib and lapatinib.

The ligands neuregulin 1 (NRG) or neuregulin 2 bind to the extracellular domain of HER3 and activate receptor-mediated signaling pathway by promoting dimerization with other dimerization partners such as HER2. Heterodimerization results in activation and transphosphorylation of HER3's intracellular domain and is a means not only for signal diversification but also signal amplification. In addition, HER3 heterodimerization can also occur in the absence of activating ligands and this is commonly termed ligand-independent HER3 activation. For example, when HER2 is expressed at high levels as a result of gene amplification (e.g. breast, lung, ovarian or gastric cancer) spontaneous HER2/HER3 dimers can be formed. In this situation the HER2/HER3 is considered the most active ErbB signaling dimer and is therefore highly transforming.

It has previously been shown that also HER3 is mutated in ˜11% of colon and gastric cancers which promotes oncogenic signaling in presence of HER2 (Jaiswal et al., 2013, Oncogenic ErbB3 mutations in human cancers. Cancer Cell 23, 603-617). These gain-of-function mutations in the HER3 pseudokinase domain enhance the allosteric activator potential of HER3.

Heterodimerization of HER3 with EGFR or HER2 also plays a role in oncogenic signaling by the HER family and contributes to cellular mechanisms that cause resistance to cancer therapeutics targeting EGFR and HER2. Heterodimerization results in activation of the ErbB receptor kinase domain and cross-phosphorylation of the ErbB receptors, which is known to occur between, e.g., EGFR and HER2, HER2 and ErbB3, and HER2 and ErbB4, and EGFR and ErbB3. The design of next-generation inhibitors that could overcome this developed resistance is now focused on directly targeting HER3 or HER3-containing heterodimers

Markedly elevated levels of HER3 have been found in several types of cancer such as breast, lung, gastrointestinal and pancreatic cancers indicating that ErbB3, like EGFR and HER2, plays a role in human malignancies. Interestingly, a correlation between the expression of HER2/HER3 and the progression from a non-invasive to an invasive stage has been shown (Alimandi et al, (1995) Oncogene 10:1813-1821; DeFazio et ai, (2000) Cancer 87:487-498; Naidu et al, (1988) Br. J. Cancer 78: 1385-1390). Accordingly, agents that interfere with HER3 mediated signaling are needed.

ErbB family members can be targeted with antibodies. They can inhibit ligand binding and/or receptor dimerization. Furthermore, antibodies can induce receptor internalization and degradation by receptor crosslinking (Friedman et al, 2005, PNAS 102: 1915-1920; Roepstorff et al, 2008, Histochem Cell Biol. 129:563-578; Moody et al., 2015, Mol. Therapy 23: 1888-1898). Additionally, antibodies containing an Fc part can mediate cancer cell killing through effector functions like antibody-dependent cellular cytotoxicity (ADCC) and complement-dependent cytotoxicity (CDC). Antibodies can also be used as delivery system for cytotoxic agents to cancer cells. Because of its emerging role as heterodimerization partner involved in propagating tumorigenesis and the development of resistance to therapy, HER3 has become a target for antibody therapy. Various antibodies directed against HER3 have been developed (Gaborit et al. 2015, Hum. Vaccin. Immunother. 12: 576-592; Dey et al. 2015, Am. J. Transl. Res. 7: 733-750; Aurisicchio et al. 2012, Oncotarget 3, 744-758; Baselga & Swain 2009, Nat. Rev. Cancer 9: 463-475; Gala & Chandariapaty 2014, Clin. Cancer Res. 20: 1410-1416; Kol et al. 2014, Pharmacol. Ther. 143: 1-11; Zhang et al. 2016, Acta Biochim. Biophys. Sin. 48: 39-48).

The complex mechanisms regulating the function of HER3 warrant further research on new and optimized therapeutic strategies against this protein. Accordingly, there remains a need for developing novel, effective and safe products that modulate the activity of HER3 and thus treat HER3-related diseases, such as cancer.

### SUMMARY OF THE INVENTION

The invention is based on the discovery of antibodies or fragments thereof that bind to extracellular region (ectodomain) of HER3 receptor and block both ligand-dependent (e.g. neuregulin) and ligand-independent HER3 signaling pathways. The invention is also based on the discovery of antibodies or fragments thereof that bind to amino acid residues within ectodomain of HER3 and block both ligand-dependent (e.g. neuregulin) and ligand-independent HER3 signaling pathways.

In another aspect, the invention pertains to isolated antibody or fragment thereof that recognizes an epitope of a HER3 receptor, wherein the epitope comprises amino acid residues within ectodomain of the HER3 receptor, and wherein the antibody or fragment thereof blocks both ligand-dependent and ligand-independent signal transduction.

In another aspect, the invention pertains to an isolated antibody or fragment thereof to a HER3 receptor, having a dissociation (K_{D}) of at least 1×10⁻⁷ M, 10⁻⁸ M, 10⁻⁹ M, 10⁻¹⁰ M, 10⁻¹¹ M, 10⁻¹² M, 10⁻¹³ M, wherein the antibody or fragment thereof blocks both ligand-dependent and ligand-independent signal transduction.

In another aspect, the invention pertains to a fragment of an antibody that binds to HER3 selected from the group consisting of; Fab, F(ab₂)′, F(ab)₂′, scFv, VHH, VH, VL, dAbs, wherein the fragment of the antibody blocks both ligand-dependent and ligand-independent signal transduction.

The antigen-binding protein that binds to HER3 can be an antibody. The antibody can be a monoclonal antibody, a polyclonal antibody, a recombinant antibody, a humanized antibody, a human antibody, a chimeric antibody, a multi-specific antibody, or an antibody fragment thereof (e.g., a Fab fragment, a Fab′ fragment, a F(ab′)2 fragment, a Fv fragment, a diabody, or a single chain antibody molecule). The antibody can be of the IgGI-, IgG2-, IgG3- or IgG4-type.

In another aspect, the invention pertains to a pharmaceutical composition comprising an antibody or fragment thereof and a pharmaceutically acceptable carrier. In one embodiment, the pharmaceutical composition further comprises an additional therapeutic agent. In one embodiment, the additional therapeutic agent is selected from the group consisting of an EGFR inhibitor, a HER2 inhibitor, a HER3 inhibitor, a HER4 inhibitor, an mTOR inhibitor and a PI3 Kinase inhibitor. In one embodiment, the additional therapeutic agent is a EGFR inhibitor selected from the group consisting of Matuzumab (EMD72000), Erbitux®/Cetuximab, Vectibix®/Panitumumab, mAb 806, Nimotuzumab, Iressa®/Gefitinib, CI-1033 (PD183805), Lapatinib (GW-572016), Tykerb®/Lapatinib Ditosylate, Tarceva®/Erlotinib HCL (OSI-774), PKI-166, and Tovok®; a HER2 inhibitor selected from the group consisting of Pertuzumab, Trastuzumab, MM-111, neratinib, lapatinib or lapatinib ditosylate/Tykerb®; a HER3 inhibitor selected from the group consisting of, MM-121, MM-111, IB4C3, 2DID12 (U3 Pharma AG), AMG888 (Amgen), AV-203 (Aveo), MEHD7945A (Genentech), MOR10703 (Novartis) and small molecules that inhibit HER3; and a HER4 inhibitor. In one embodiment, the additional therapeutic agent is a HER3 inhibitor, wherein the HER3 inhibitor is MORI 0703. In one embodiment, the additional therapeutic agent is an mTOR inhibitor selected from the group consisting of Temsirolimus/Torisel®, ridaforolimus/Deforolimus, AP23573, MK8669, everolimus/Affinitor®. In one embodiment, the additional therapeutic agent is a PI3 Kinase inhibitor selected from the group consisting of GDC 0941, BEZ235, BKM120 and BYL719.

In one aspect, the invention pertains to a method of treating a cancer comprising selecting a subject having an HER3 expressing cancer, administering to the subject an effective amount of a composition comprising an antibody or fragment thereof disclosed herein. In one embodiment, the subject is a human and the cancer is selected from the group consisting of breast cancer, colorectal cancer, lung cancer, pancreatic ductal adenocarcinoma, multiple myeloma, ovarian cancer, liver cancer, gastric cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, and melanoma, prostate cancer, benign prostatic hyperplasia, gynacomastica, and endometriosis.

In one aspect, the invention pertains to a method of treating a cancer comprising selecting a subject having NRG1-rearranged fusions expressing cancer, administering to the subject an effective amount of a composition comprising an antibody or fragment thereof disclosed herein. In one embodiment, the subject is a human and the cancer is selected from the group consisting of breast cancer, colorectal cancer, lung cancer, pancreatic ductal adenocarcinoma, multiple myeloma, ovarian cancer, liver cancer, gastric cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, and melanoma, prostate cancer, benign prostatic hyperplasia, gynacomastica, and endometriosis.

In one aspect, the invention pertains to use of the antibody or fragment thereof for treating subjects having an HER3 associated disease, by administering an agent that binds to HER3, in combination with a second agent that binds to and/or inhibits another member of the HER family. The first and the second agent may be any kind of molecule that binds to HER3 or binds to and/or inhibits another HER family member, respectively, including, but not limited to a biological compound, such as an antigen binding protein, a small molecular tyrosine kinase inhibitor, antisense oligonucleotides, an siRNA, or a natural substance.

In one aspect, the invention features a method of treating or preventing a disease associated with HER3 in a subject, comprising administering to the subject a first agent and a second agent, wherein the first agent binds to HER3 and the second agent binds to and/or inhibits the activity of another member of the HER family. The first agent can be a small molecule compound or an antigen-binding protein that binds to HER3. The first agent can be an antigen-binding protein that binds to HER3 and comprises a heavy chain amino acid sequence that comprises a VH CDR1 consisting of SEQ ID NO: 3; a VH CDR2 consisting of SEQ ID NO: 4; and a VH CDR3 consisting of SEQ ID NO: 5; and a light chain amino acid sequence that comprises a VL CDR1 consisting of SEQ ID NO: 6; a VL CDR2 consisting of SEQ ID NO: 7; and a VL CDR3 consisting of SEQ ID NO: 8. The first agent can be an antigen-binding protein that binds to HER3 and comprises a heavy chain amino acid sequence that comprises at least one of the CDR's consisting of (a) VH CDR1 consisting of SEQ ID NO: 3; (b) VH CDR2 consisting of SEQ ID NO: 4; and (c) VH CDR3 consisting of SEQ ID NO: 5. The first agent can be an antigen-binding protein that binds to HER3 and comprises a light chain amino acid sequence that comprises at least one of the CDR's selected from the group consisting of: (d) VL CDR1 consisting of SEQ ID NO: 6; (e) VL CDR2 consisting of SEQ ID NO: 7; and (f) VL CDR3 consisting of SEQ ID NO: 8.

The first agent can be an antigen-binding protein that binds to HER3 and comprises a heavy chain amino acid sequence that comprises at least one of the CDR's selected from the group consisting of (a) VH CDR1 consisting of SEQ ID NO: 3; (b) VH CDR2 consisting of SEQ ID NO: 4 and (c) VH CDR3 consisting of SEQ ID NO: 5; and a light chain amino acid sequence that comprises at least one of the CDR's selected from the group consisting of: (d) VL CDR1 consisting of SEQ ID NO: 6; (e) VL CDR2 consisting of SEQ ID NO: 7; and (f) VL CDR3 consisting of SEQ ID NO: 8. The first agent can be an antigen-binding protein that binds to HER3 and comprises a heavy chain amino acid sequence that comprises a VH CDR1 consisting of SEQ ID NO: 3, a VH CDR2 consisting of SEQ ID NO: 4, and a VH CDR3 consisting of SEQ ID NO: 5, or a light chain amino acid sequence that comprises a VL CDR1 consisting of SEQ ID NO: 6, a VL CDR2 consisting of SEQ ID NO: 7, and VL CDR3 consisting of SEQ ID NO: 8.

The first agent can be an antigen-binding protein that binds to HER3 and comprises a heavy chain amino acid sequence consisting of SEQ ID NO: 1. The antigen-binding protein can include a light chain amino acid sequence consisting of SEQ ID NO: 2.

The first agent can be an antigen-binding protein that binds to HER3 and comprises a heavy chain amino acid sequence consisting of SEQ ID NO: 1 and a light chain amino acid sequence consisting of SEQ ID NO: 2.

The first agent can be an antigen-binding protein that binds to HER3, and the antigen-binding protein can be coupled to an effector group. The effector group can be a radioisotope or radionuclide, a toxin, or a therapeutic or chemotherapeutic group (e.g., a therapeutic or chemotherapeutic group selected from the group consisting of calicheamicin, auristatin-PE, geldanamycin, maytansine and derivatives thereof).

The second agent can be a small molecule compound or an antigen-binding protein. The second agent can be, for example, trastuzumab, lapatinib, neratinib, panitumumab, erlotinib, cetuximab, pertuzumab, and T-DM1.

In another aspect, this document features a method of treating or preventing a disease associated with HER3 in a subject, comprising administering to the subject a first agent and a second agent, wherein the first agent is an antigen-binding protein that binds to HER3 and comprises the heavy chain amino acid sequence of consisting of SEQ ID NO: 1 and the light chain amino acid sequence of consisting of SEQ ID NO: 2, and wherein the second agent is selected from the group consisting of erlotinib, lapatinib, and neratinib. In addition, this document features methods of treating or preventing a disease associated with HER3 in a subject, comprising administering to the subject a first agent and a second agent, wherein the first agent is an antigen-binding protein that binds to HER3 and comprises the heavy chain amino acid sequence of consisting of SEQ ID NO: 1 and the light chain amino acid sequence of consisting of SEQ ID NO: 2 or an antigen-binding protein that binds to HER3 and comprises the heavy chain amino acid sequence of consisting of SEQ ID NO: 1 and the light chain amino acid sequence of consisting of SEQ ID NO: 2, and wherein the second agent is selected from the group consisting of erlotinib, lapatinib, and neratinib.

This invention also features a method of treating or preventing a disease associated with HER3 in a subject, comprising administering to the subject a first agent and a second agent, wherein the first agent is an antigen-binding protein that binds to HER3 and comprises the heavy chain amino acid sequence of consisting of SEQ ID NO: 1 and the light chain amino acid sequence of consisting of SEQ ID NO: 2, and wherein the second agent is selected from the group consisting of trastuzumab, T-DM1, panitumumab, and cetuximab.

The methods provided herein can optionally include administering a third or further therapeutic agent and/or radiation therapy. The third or further therapeutic agent can be an anti-neoplastic agent (e.g., an anti-tumor antibody or a chemotherapeutic agent, such as capecitabine, anthracycline, doxorubicin, cyclophosphamide, paclitaxel, docetaxel, cisplatin, gemcitabine, or carboplatin).

The first agent and the second agent can be administered by intravenous, subcutaneous, intramuscular or oral administration. The disease can be a hyperproliferative disease (e.g., a disease selected from the group consisting of breast cancer, ovarian cancer, prostate cancer, colon cancer, renal cancer, lung cancer, pancreatic cancer, epidermoid carcinoma, fibrosarcoma, melanoma, nasopharyngeal carcinoma, and squamous cell carcinoma).

### BRIEF DESCRIPTION OF THE DRAWINGS

The details of one or more embodiments of the invention are set forth in the accompanying drawings and the description below. Other features, objects, and advantages of the invention will be apparent from the description and drawings, and from the claims.

FIG. 1: provides nucleotide sequences encoding light chain variable domains and heavy chain variable domains.

FIG. 2: provides amino acid sequences of light chain variable domain. CDR and FR regions are indicated.

FIG. 3: provides amino acid sequences of heavy chain variable domain. CDR and FR regions are indicated.

FIG. 4: provides amino acid sequences of the light chain CDR1 region of light chain variable domains.

FIG. 5: provides amino acid sequences of the light chain CDR2 regions of light chain variable domains.

FIG. 6: provides amino acid sequences of the light chain CDR3 regions of light chain variable domains.

FIG. 7: provides amino acid sequences of the heavy chain CDR1 regions of heavy chain variable domains.

FIG. 8: provides amino acid sequences of the heavy chain CDR2 regions of heavy chain variable domains.

FIG. 9: provides amino acid sequences of the heavy chain CDR3 regions of heavy chain variable domains.

FIG. 10: Binding specificity of 29Z6 antibody to Human HER3. Western blot shows specific band was detected for HER3 but not EGFR or HER2.

FIG. 11: Binding kinetics of 29Z6 monoclonal antibody to Human HER3 by Surface plasmon resonance.

FIG. 12: provides graphs illustrating the ability of 29Z6 anti-HER3 antibody to inhibit the growth of BT-474 cells.

FIG. 13: provides graphs illustrating the ability of 29Z6 anti-HER3 antibody to inhibit the growth of FaDu cells.

FIG. 14: provides graphs illustrating the ability of 29Z6 anti-HER3 antibody to inhibit the growth of MDA-MB231 cells.

FIG. 15: provides graphs illustrating the ability of 29Z6 anti-HER3 antibody to inhibit the growth of BxPC3 Luc cells.

FIG. 16: provides graphs illustrating the ability of 29Z6 anti-HER3 antibody to inhibit the growth of A549 cells.

FIG. 17: 29Z6 anti-HER3 antibody suppresses proliferation and colony formation in FaDu cells

FIG. 18: 29Z6 anti-HER3 antibody suppresses proliferation and colony formation in BT-474 cells

FIG. 19: 29Z6 anti-HER3 antibody suppresses proliferation and colony formation in PANC-1 cells.

FIG. 20: 29Z6 anti-HER3 antibody suppresses proliferation and colony formation in MCF-7 cells.

FIG. 21: 29Z6 anti-HER3 antibody suppresses proliferation and colony formation in SK-BR-3 cells.

FIG. 22: shows a reduction in tumor volume after administration of 29Z6 using the human Bx-PC3 pancreatic cancer xenograft model.

### DESCRIPTION OF THE SEQUENCE LISTING

SEQ ID NO: 1, 29Z6 Heavy Chain—amino acids sequence

SEQ ID NO: 2, 29Z6 Light Chain—Amino acids sequence

SEQ ID NO: 3 29Z6 Heavy Chain CDR1—amino acids sequence

SEQ ID NO: 4 29Z6 Heavy Chain CDR2—amino acids sequence

SEQ ID NO: 5 29Z6 Heavy Chain CDR3—amino acids sequence

SEQ ID NO: 6 29Z6 Light Chain CDR1—Amino acids sequence

SEQ ID NO: 7 29Z6 Light Chain CDR2—Amino acids sequence

SEQ ID NO: 8 29Z6 Light Chain CDR3—Amino acids sequence

SEQ ID NO: 9 29Z6 Heavy Chain—DNA sequence

SEQ ID NO: 10 29Z6 Light Chain—DNA sequence

SEQ ID NO: 11 29Z6 Heavy Chain CDR1—DNA sequence

SEQ ID NO: 12 29Z6 Heavy Chain CDR2—DNA sequence

SEQ ID NO: 13 29Z6 Heavy Chain CDR3—DNA sequence

SEQ ID NO: 14 29Z6 Light Chain CDR1—DNA sequence

SEQ ID NO: 15 29Z6 Light Chain CDR2—DNA sequence

SEQ ID NO: 16 29Z6 Light Chain CDR3—DNA sequence

### DETAILED DESCRIPTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Definitions

The term “HER3” or “HER3 receptor” also known as “ErbB3” as used herein refers to mammalian HER3 protein and “her3” or “erbB3” refers to mammalian her3 gene. The preferred HER3 protein is human HER3 protein present in the cell membrane of a cell. The human her3 gene is described in U.S. Pat. No. 5,480,968 and Plowman et ah, (1990) PNAS, 87:4905-4909.

Human HER3 as defined in Accession No. NP_001973 (human), and represented below. All nomenclature is for full length, immature HER3 (amino acids 1-1342). The immature HER3 is cleaved between positions 19 and 20, resulting in the mature HER3 protein (20-1342 amino acids).

mrandalqvl gllfslargs evgnsqavcp gtlnglsvtg
daenqyqtly klyercewm gnleivltgh nadlsflqwi
revtgyvlva mnefstlplp nlrwrgtqv ydgkfaifvm
lnyntnssha lrqlrltqlt eilsggvyie kndklchmdt
idwrdivrdr daeiwkdng rscppchevc kgrcwgpgse
dcqtltktic apqcnghcfg pnpnqcchde caggcsgpqd
tdcfacrhfn dsgacvprcp qplvynkltf qlepnphtky
qyggvcvasc phnfwdqts cvracppdkm evdknglkmc
epcgglcpka cegtgsgsrf qtvdssnidg fvnctkilgn
ldflitglng dpwhkipald peklnvfrtv reitgylniq
swpphmhnfs vfsnittigg rslynrgfsl limknlnvts
lgfrslkeis agriyisanr qlcyhhslnw tkvlrgptee
rldikhnrpr rdcvaegkvc dplcssggcw gpgpgqclsc
rnysrggvcv thcnflngep refaheaecf schpecqpme
gtatcngsgs dtcaqcahfr dgphcvsscp hgvlgakgpi
ykypdvqnec rpchenctqg ckgpelqdcl gqtlvligkt
hltmaltvia glwifmmlg gtflywrgrr iqnkramrry
lergesiepl dpsekankvl arifketelr klkvlgsgvf
gtvhkgvwip egesikipvc ikviedksgr qsfqavtdhm
laigsldhah ivrllglcpg sslqlvtqyl plgslldhvr
qhrgalgpql llnwgvqiak gmyyleehgm vhrnlaarnv
llkspsqvqv adfgvadllp pddkqllyse aktpikwmal
esihfgkyth qsdvwsygvt vwelmtfgae pyaglrlaev
pdllekgerl aqpqictidv ymvmvkcwmi denirptfke
laneftrmar dpprylvikr esgpgiapgp ephgltnkkl
eevelepeld ldldleaeed nlatttlgsa lslpvgtlnr
prgsqsllsp ssgympmnqg nlgescqesa vsgssercpr
pvslhpmprg clasessegh vtgseaelqe kvsmcrsrsr
srsprprgds ayhsqrhsll tpvtplsppg leeedvngyv
mpdthlkgtp ssregtlssv glssvlgtee ededeeyeym
nrrrrhspph pprpssleel gyeymdvgsd lsaslgstqs
cplhpvpimp tagttpdedy eymnrqrdgg gpggdyaamg
acpaseqgye emrafqgpgh qaphvhyarl ktlrsleatd
safdnpdywh srlfpkanaq rt

The term “HER3 ligand” as used herein refers to polypeptides which bind and activate HER3. Examples of HER3 ligands include, but are not limited to neuregulin 1 (NRG) and neuregulin 2, betacellulin, heparin-binding epidermal growth factor, and epiregulin. The term includes biologically active fragments and/or variants of a naturally occurring polypeptide.

The “HER2-HER3 protein complex” is a noncovalently associated oligomer containing HER2 receptor and the HER3 receptor. This complex can form when a cell expressing both of these receptors is exposed to a HER3 ligand e.g., NRG or when HER2 is active/overexpressed

The phrase “HER3 activity” or “HER3 activation” as used herein refers to an increase in oligomerization (e.g. an increase in HER3 containing complexes), HER3 phosphorylation, conformational rearrangements (for example those induced by ligands), and HER3 mediated downstream signaling.

The term “disease associated with HER3 dependent signaling,” “HER3 related disorder,” “disorder associated with HER3 dependent signaling,” “HER3 dependent disorder,” or “HER3 signaling dependent disorder” as used herein, includes disease states and/or symptoms associated with a disease state, where increased levels of HER3 and/or activation of cellular cascades involving HER3 are found. It is understood that HER3 heterodimerizes with other ErbB proteins such as, EGFR and HER2, when increased levels of HER3 are found. Accordingly, the term “disease associated with HER3 dependent signaling,” also includes disease states and/or symptoms associated with disease states where increased levels of EGFR/HER3 and/or HER2/HER3 and/or HER3/ErbB4 heterodimers are found. In general, the term “disease associated with HER3 dependent signaling,” refers to any disorder, the onset, progression or the persistence of the symptoms of which requires, or is influenced by the participation of HER3. Exemplary HER3-mediated disorders include, but are not limited to, for example, cancer.

The phrase “inhibition of proliferation of a cell expressing HER3,” as used herein, refers to the ability of an antigen binding portion thereof, and/or antibody to statistically significantly decrease proliferation of a cell expressing HER3 relative to the proliferation in the absence of the antibody. In some embodiments, the proliferation of a cell expressing HER3 (e.g., a cancer cell) can be decreased by at least 10%, or at least 20%, or at least 30%, or at least 40%, or at least 50%, or at least 60%, or at least 70%, or at least 80%, or at least 90%, or at least 91, 92, 93, 94, 95, 96, 97, 98, 99%, or 100% when the cells are contacted with an antibody of the present disclosure, relative to the proliferation measured in the absence of a negative control antibody. Cellular proliferation can be assayed using art recognized techniques which measure cell number and/or rate of cell division, the fraction of cells within a cell population undergoing cell division, and/or rate of cell loss from a cell population due to terminal differentiation or cell death (e.g., using CyQUANT Cell Proliferation Assay or CellTiterGlo assay).

The term “identical” in the context of two or more nucleic acids or polypeptide sequences, refers to two or more sequences or subsequences that are the same, i.e. comprise the same sequence of nucleotides or amino acids. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96% o, at least 97%, at least 98%>, or at least 99% identity over a specified region), when compared and aligned for maximum correspondence over a comparison window, or designated region as measured using one of the following sequence comparison algorithms or by manual alignment and visual inspection. An “isolated antibody,” as used herein, is intended to refer to an antibody which is substantially free of other antibodies having different antigenic specificities (e.g., an isolated antibody that specifically binds to HER3 is substantially free of antibodies that specifically bind antigens other than HER3). In addition, an isolated antibody is typically substantially free of other cellular material and/or proteins. As used herein, “isotype” refers to the antibody class (e.g., IgM or IgG1) or antibody that is encoded by heavy chain constant region genes. In some embodiments, an antibody or antigen binding portion thereof is of an isotype selected from an IgG1, an IgG2, an IgG3, an IgG4, an IgM, an IgA1, an IgA2, an IgAsec, an IgD, or an IgE antibody isotype. In some embodiments, an antibody is of the IgG1 isotype. In some embodiments, an antibody is of the IgG2 isotype.

The term “antigen-binding protein”, as used herein, refers to immunoglobulin molecules and immunologically active portions of immunoglobulin molecules, i.e. molecules that contain an antigen-binding site that immunospecifically binds an antigen. Also comprised are immunoglobulin-like proteins that are selected through techniques including, for example, phage display to specifically bind to a target molecule or target epitope. In assessing the binding and/or specificity of an antigen binding protein, e.g., an antibody or immunologically functional fragment thereof, an antibody or fragment can substantially inhibit binding of a ligand to its binding partner when an excess of antibody reduces the quantity of binding partner bound to the ligand by at least about 1-20, 20-30%, 30-40%, 40-50%, 50-60%, 60-70%, 70-80%, 80-85%, 85-90%, 90-95%, 95-97%, 97-98%, 98-99% or more (e.g. as measured in an in vitro competitive binding assay). The neutralizing ability may be described in terms of an IC50 or EC50 value.

The phrase “inhibiting antibody” as used herein refers to an antibody that binds with HER3 and inhibits the biological activity of HER3 signaling, e.g., reduces, decreases and/or inhibits HER3 induced signaling activity, e.g., in a phospho-HER3 or phospho-Akt assay. Examples of assays are described in more details in the examples below. Accordingly, an antibody that “inhibits” one or more of these HER3 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits HER3 activity effects such a statistically significant decrease by at least 10% of the measured parameter, by at least 50%>, 80%> or 90%>, and in certain embodiments an antibody of the invention may inhibit greater than 95%, 98% or 99% of HER3 functional activity as evidenced by a reduction in the level of cellular HER3 phosphorylation.

Various aspects of the invention are described in further detail in the following sections and subsections.

### EMBODIMENTS

In particular embodiments of the present invention, the antigen binding protein is an antibody which is selected from the group consisting of a polyclonal antibody, a monoclonal antibody, monovalent antibodies, bispecific antibody, heteroconjugate antibodies, multispecific antibodies, deimmunized antibodies a chimeric antibody, a humanized antibody, and a human antibody (in particular a human IgGI antibody).

In particular embodiments, the antigen-binding fragment of the antibody is selected from the group consisting of a Fab fragment, a Fab′ fragment, a F(ab′)₂ fragment, a Fd fragment, a Fv fragment, a disulfide-linked Fv (dsFv), a single domain antibody, a single chain Fv (scFv) antibody, and a single domain antibody (VH, VL, VHH, Nanobody, Shark Variable New Antigen Receptor).

In particular embodiments, the antibody-like protein is selected from the group consisting of lipoprotein-associated coagulation inhibitor (LACI-D1); affilins, e.g. human-γ B crystalline or human ubiquitin; cystatin; Sac7D from Sulfolobus acidocaldarius; lipocalin and anticalins derived from lipocalins; designed ankyrin repeat domains (DARPins); SH3 domain of Fyn; Kunits domain of protease inhibitors; monobodies, e.g. the 10^{th} type III domain of fibronectin; adnectins; cysteine knot miniproteins; atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II; affilins; armadillo repeat proteins.

In particular embodiments, the antigen binding protein is monospecific, bispecific or multispecific. In particular embodiments, the bispecific or multispecific antigen binding protein specifically binds to a second cellular target. In particular embodiments, the second cellular target is selected from the group consisting of a protein expressed on the surface of an immune cell, preferably CD3, a protein expressed on the surface of tumor cells, in particular the extracellular region of a growth receptor, in particular EGFR, HER2, HER4, insulin-like growth factor 1-receptor (IGF-1R), hepatocyte growth factor receptor (HGFR, c-MET), and derivatives thereof, in particular EGFR or HER2.

In particular embodiments, the antigen binding protein is tri- or tetravalent. In particular embodiments, the antigen binding protein comprises an effector domain which is in particular bound by Fc receptors, neonatal Fc receptor (FcRn) or the complement system. In particular embodiments, the Fc domain is an domain bound by Fc gamma receptors, in particular by CD16, CD32, and/or CD64. In particular embodiment, the Fc domain is a domain activating the complement system, in particular by binding to Cl q of the complement system.

In a preferred embodiment of the present invention, the antigen binding protein is bivalent. In particular embodiments of the present invention, the antigen binding protein comprises a variable domain comprising a heavy chain according to SEQ ID NO:1 or variants thereof having at least 80%, preferably 90%, more preferably at least 95% identity to amino acid sequence according to SEQ ID NO: 1.

In particular embodiments of the present invention, the antigen binding protein comprises a variable domain comprising a light chain according to SEQ ID NO: 2 or variants thereof having at least 80%, preferably 90%, more preferably at least 95% identity to amino acid sequence according to SEQ ID NO: 2.

In particular embodiments of the present invention, the antigen binding protein comprises a variable domain comprising a heavy chain according to SEQ ID NO: 1 or variants thereof having at least 80%, preferably 90%, more preferably at least 95% identity to amino acid sequence according to SEQ ID NO: 1 and a light chain according to SEQ ID NO: 2 or variants thereof having at least 80%, preferably 90%, more preferably at least 95% identity to amino acid sequence according to SEQ ID NO: 2.

It will be appreciated by those skilled in the art that in particular the sequences of the CDR, hypervariable and variable regions can be modified without losing the ability to bind HER3. For example, CDR regions will be either identical or highly homologous to the regions specified herein. By “highly homologous” it is contemplated that from 1 to 5, preferably from 1 to 4, such as 1 to 3 or 1 or 2 substitutions, deletions, or additions may be made in the CDRs. In addition, the hypervariable and variable regions may be modified so that they show substantial homology with the regions specifically disclosed herein.

Furthermore, it may be desired according to the present invention to modify the amino acid sequences described herein, in particular those of human heavy chain constant regions to adapt the sequence to a desired allotype, e.g. an allotype found in the Caucasian population.

It may be desired according to the present invention to modify the antibodies in in the Fc region in order to change the functional or pharmacokinetic properties of the antibodies. Such alterations may result in a decrease or increase of Clq binding and CDC or of FcyR binding and ADCC. Substitutions can, for example, be made in one or more of the amino acid residues of the heavy chain constant region, thereby causing an alteration in an effector function while retaining the ability to bind to the antigen as compared with the modified antibody, cf. U.S. Pat. Nos. 5,624,821 and 5,648,260.

Furthermore, the glycosylation pattern of antibodies can be modified in order to change the effector function of the antibodies which enhances the affinity of the Fc region for Fc-Receptors which, in turn, will result in an increased ADCC of the antibodies in the presence of NK cells. Furthermore, modification of galactosylation can be made in order to modify CDC.

In one aspect, the present invention provides isolated antibodies that specifically bind to human HER3 protein, the antibodies comprising a VH domain having an amino acid sequence of SEQ ID NO: 1.

Accordingly, in one aspect, the invention provides an isolated monoclonal antibody or fragment thereof having: a heavy chain variable region comprising an amino acid sequence selected from the group consisting of SEQ ID NO: 1; and a light chain variable region comprising an amino acid sequence selected from the group consisting of SEQ ID NOs: 2; wherein the antibody specifically binds to human HER3 protein.

In another aspect, the present invention provides isolated HER3 antibodies that bind to human HER3 protein that comprise the heavy chain and light chain CDR1, CDR2 and CDR3 or combinations thereof. The amino acid sequence of the VH CDR1 of the antibody is shown in SEQ ID NO: 3. The amino acid sequence of the VH CDR2 of the antibody is shown in SEQ ID NO: 4. The amino acid sequence of the VH CDR3 of the antibody is shown in SEQ ID NO: 5. The amino acid sequence of the VL CDRI of the antibody is shown in SEQ ID NO: 6. The amino acid sequence of the VL CDR2 of the antibody is shown in SEQ ID NO: 7. The amino acid sequence of the VL CDR3 of the antibody is shown in SEQ ID NO: 8. The CDR regions are delineated using the Kabat system (Kabat et αi, (1991) Sequences of Proteins of Immunological Interest, Fifth Edition, U.S. Department of Health and Human Services, NIH Publication No. 91-3242; Chothia et al, (1987) J. Mol. Biol. 196:901-917; Chothia et al, (1989) Nature 342: 877-883; and Al-Lazikani et al, (1997) J. Mol. Biol. 273, 927-948).

The antibodies disclosed herein can be derivatives of single chain antibodies, diabodies, domain antibodies, nanobodies, and unibodies. For example, the invention provides an isolated monoclonal antibody (or a functional fragment thereof) comprising a heavy chain variable region and a light chain variable region, wherein the heavy chain variable region comprises an amino acid sequence that is at least 80%, 90%, 95%, 96%, 97%, 98% or 99% identical to an amino acid sequence consisting of SEQ ID NO: 1; the light chain variable region comprises an amino acid sequence that is at least 80%>, 90%>, 95%, 96%, 97%), 98% o or 99% identical to an amino acid sequence consisting of SEQ ID NO: 2; wherein the antibody binds to HER3 and inhibits the signaling and function activity of HER3, which can be measured by various methods such as phosphorylation assay or cell proliferation, and ligand blocking assays. Also includes within the scope of the invention are variable heavy and light chain parental nucleotide sequences; and full length heavy and light chain sequences optimized for expression in a mammalian cell.

In other embodiments, the variable regions of heavy chain and/or light chain nucleotide sequences may be 60%, 70%, 80%, 90%, 95%, 96%, 97%, 98% or 99% identical to the sequences set forth above.

In certain embodiments, an antibody of the invention has a heavy chain variable region comprising CDRI, CDR2, and CDR3 sequences and a light chain variable region comprising CDRI, CDR2, and CDR3 sequences, wherein one or more of these CDR sequences have specified amino acid sequences based on the antibody described herein or conservative modifications thereof, and wherein the antibodies retain the desired functional properties of the HER3 antibody of the invention.

In one embodiment, the antibody or fragments thereof binds to HER3 and inhibits both ligand dependent and ligand-independent HER3 signal transduction. In one embodiment, the antibody or fragments thereof bind to HER3 and inhibits both ligand dependent and ligand-independent HER3 signal transduction.

Consequently, the antibody 29Z6 of the present invention may be used to treat conditions where existing therapeutic antibodies are clinically ineffective.

In one embodiment provided a method of treating a cancer, comprising: testing a subject, e.g., a sample (e.g., a subject's sample comprising cancer cells) for the presence of the antibody of the invention, wherein said anti-human Her3 antibody or fragment inhibits NRG1-rearranged cancers.

In particular embodiment, the antibody of the present invention, wherein said anti-human Her3 antibody or fragment inhibits cancers with one or more of NRG1-rearranged fusions: (Cluster of Differentiation 74-Neuregulin-1) CD74-NRG1 fusion, (Solute Carrier Family 3 Member 2-Neuregulin-1) SLC3A2-NRG1 fusion, (Syndecan-4-Neuregulin-1) SDC4-NRG1 fusion, DOC4-NRG1 fusion, (Rho-associated protein kinase 1-Neuregulin-1) ROCK1-NRG1 fusion, (Forkhead Box A1-Neuregulin-1) FOXA1-NRG1 fusion, (A-Kinase Anchoring Protein 13-Neuregulin-1) AKAP13-NRG1 fusion, (Thrombospondin 1-Neuregulin-1) THBS1-NRG1 fusion, (Phosphodiesterase 7A-Neuregulin-1) PDE7A-NRG1 fusion, (ATPase Na+/K+ Transporting Subunit Beta 1-Neuregulin-1) ATP1B1-NRG1 fusion, NRG1-PMEPA1 fusion, Clusterin-NRG1 fusion.

In particular embodiment, a patient stratification method where tumors are screened first for NRG1-rearranged fusions and then patients with positive NRG1-rearranged fusions are treated with anti-human Her3 antibody of the present invention.

### Engineered and Modified Antibodies

An antibody of the invention further can be prepared using an antibody having one or more of the VH and/or VL sequences shown herein as starting material to engineer a modified antibody, which modified antibody may have altered properties from the starting antibody.

An antibody can be engineered by modifying one or more residues within one or both variable regions (i. e., VH and/or VL), for example within one or more CDR regions and/or within one or more framework regions. Additionally or alternatively, an antibody can be engineered by modifying residues within the constant region(s), for example to alter the effector function(s) of the antibody.

Grafting Antibody Fragments into Alternative Frameworks or Scaffolds

A wide variety of antibody/immunoglobulin frameworks or scaffolds can be employed so long as the resulting polypeptide includes at least one binding region which specifically binds to HER3. Such frameworks or scaffolds include the 5 main idiotypes of human immunoglobulins, or fragments thereof, and include immunoglobulins of other animal species, preferably having humanized aspects.

Novel frameworks, scaffolds and fragments continue to be discovered and developed by those skilled in the art.

In one aspect, the invention pertains to generating non-immunoglobulin based antibodies using non-immunoglobulin scaffolds onto which CDRs of the invention can be grafted.

Known or future non-immunoglobulin frameworks and scaffolds may be employed, as long as they comprise a binding region specific for the target HER3 protein (e.g., human and/or cynomologus HER3). Known non-immunoglobulin frameworks or scaffolds include, but are not limited to, fibronectin, ankyrin, domain antibodies, lipocalin, small modular immuno-pharmaceuticals, maxybodies, Protein A, and affilin.

### Humanized Antibodies

Compared to monoclonal antibodies, the humanized HER3 antibodies or fragments thereof, will further reduce antigenicity when administered to human subjects.

### Camelid Antibodies

A feature of the present invention is a camelid antibody or nanobody having high affinity for HER3. In certain embodiments herein, the camelid antibody or nanobody is naturally produced in the camelid animal, i.e., is produced by the camelid following immunization with HER3 or a peptide fragment thereof, using techniques described herein for other antibodies. Alternatively, the HER3-binding camelid nanobody is engineered, i.e., produced by selection for example from a library of phage displaying appropriately mutagenized camelid nanobody proteins using panning procedures with HER3 as a target as described in the examples herein.

### Bispecific Molecules and Multivalent Antibodies

In another aspect, the present invention can be biparatopic, bispecific or multispecific molecules comprising an antibody or a fragment thereof that binds to a non-linear or conformational epitope within HER3. In another aspect, the biparatopic, bispecific or multispecific molecules comprise an antibody or a fragment thereof that binds to HER3. The antibody or fragment thereof can be derivatized or linked to another functional molecule, e.g., another peptide or protein (e.g., another antibody or ligand for a receptor) to generate a bispecific molecule that binds to at least two different binding sites or target molecules. The antibody or fragment thereof may in fact be derivatized or linked to more than one other functional molecule to generate biparatopic or multi-specific molecules that bind to more than two different binding sites and/or target molecules; such biparatopic or multi-specific molecules. To create a bispecific molecule, an antibody or fragment thereof can be functionally linked (e.g., by chemical coupling, genetic fusion, non-covalent association or otherwise) to one or more other binding molecules, such as another antibody, antibody fragment, peptide or binding mimetic, such that a bispecific molecule results. Further clinical benefits may be provided by the binding of two or more antigens within one antibody

In another embodiment, the invention pertains to dual function antibodies in which a single monoclonal antibody has been modified such that the antigen binding site binds to more than one antigen, such as a dual function antibody which binds both HER3 and another antigen (e.g., EGFR, HER2, and HER4). In another embodiment, the invention pertains to a dual function antibody that targets antigens having the same conformation, for example an antigen that has the same conformation of HER3 in the “closed” or “inactive” state. Examples of antigens with the same conformation of HER3 in the “closed” or “inactive” state include, but are not limited to, EGFR and HER4. Thus, a dual function antibody may bind to both HER3 and EGFR; HER3 and HER4, or EGFR and HER4. The dual binding specificity of the dual function antibody may further translate into dual activity, or inhibition of activity. (See e.g., Jenny Bostrom et al., (2009) Science: 323; 1610-1614).

### Antibody Conjugates

The present invention provides antibodies or fragments thereof that specifically bind to HER3 recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antibody fragment described herein (e.g., a Fab fragment, Fd fragment, Fv fragment, F(ab)2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g. antibodies or fragments thereof with higher affinities and lower dissociation rates). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a HER3 protein may be recombined with one or more components, motifs, sections, parts, domains, fragments, etc. of one or more heterologous molecules.

In other embodiments, antibodies of the present invention or fragments thereof conjugated to a diagnostic or detectable agent. Such antibodies can be useful for monitoring the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin-biotin and avidin/biotin; fluorescent materials, such as, but not limited to fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ^{m}In), technetium (⁹⁹Tc), thallium gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, 47Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; and positron emitting metals using various positron emission tomographies, and noradioactive paramagnetic metal ions.

In some embodiments, a HER3 binding protein can be coupled to an effector group. Such a binding protein can be especially useful for therapeutic applications. As used herein, the term “effector group” refers to a cytotoxic group such as a radioisotope or radionuclide, a toxin, a therapeutic group or other effector group known in the art. Examples of suitable effector groups are radioisotopes or radionuclides {e.g., ³H, ¹⁴C, ¹⁵N, ³⁵S, ⁹⁰Y, ⁹⁹Tc, ^{m}In, ¹²⁵I, ¹³¹I) or non-radio isotopes {e.g., 2D), calicheamicin, dolastatin analogs such as auristatins, and chemotherapeutic agents such as geldanamycin and maytansine derivates, including DM1. Thus, in some cases, a group can be both a labeling group and an effector group. Various methods of attaching effector groups to polypeptides or glycopolypeptides (such as antibodies) are known in the art, and may be used in making and carrying out the compositions and methods described herein. In some embodiments, it may be useful to have effector groups attached to a binding protein by spacer arms of various lengths to, for example, reduce potential steric hindrance.

The present invention further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine. In one embodiment, the HER3 antibody, or a fragment thereof is conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), ablating agents (e.g., mechlorethamine, thioepa chloraxnbucil, meiphalan, carmustine (BSNU) and lomustine (CCNU), cyclothosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics US20090304721).

Other examples of therapeutic cytotoxins that can be conjugated to an antibody or fragment thereof of the invention include duocarmycins, calicheamicins, maytansines and auristatins, and derivatives thereof. An example of a calicheamicin antibody conjugate is commercially available (Mylotarg; Wyeth-Ayerst).

Cytoxins can be conjugated to antibodies or fragments thereof of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

### Antibody Combinations

An another aspect, the invention pertains to HER3 antibodies, or fragments thereof of the invention used with other therapeutic agents such as another antibodies, small molecule inhibitors, mTOR inhibitors or PB Kinase inhibitors. Examples include, but are not limited to, the following: EGFR inhibitors: The HER3 antibodies or fragments thereof can be used with EGFR inhibitors which include, but are not limited to, Matuzumab (EMD72000), Erbitux®/Cetuximab (Imclone), Vectibix®/Panitumumab (Amgen), mAb 806, and Nimotuzumab (TheraCIM), Iressa®/Gefitinib (Astrazeneca); CI-1033 (PD183805) (Pfizer), Lapatinib (GW-572016) (Glaxo SmithKline), Tykerb®/Lapatinib Ditosylate (SmithKlineBeecham), Tarceva®/Erlotinib HCL (OSI-774) (OSI Pharma), and PKI-166 (Novartis), and N-[4-[(3-Chloro-4-fluorophenyl)amino]-7-[[(3″S″)-tetrahydro-3-furanyl]oxy]-6-quinazolinyl]-4(dimethylamino)-2-butenamide, sold under the tradename Tovok® by Boehringer Ingelheim).

HER2 inhibitors: The HER3 antibodies or fragments thereof can be used with HER2 inhibitors which include, but are not limited to, Pertuzumab (sold under the trademark Omnitarg®, by Genentech), Trastuzumab (sold under the trademark Herceptin® by Genentech/Roche), MM-111, neratinib (also known as HKI-272, (2E)-N-[4-[[3-chloro-4-[(pyridin-2-yl)methoxy]phenyl] amino]-3-cyano-7-ethoxyquinolin-6-yl]-4-(dimethylamino)but-2-enamide, and described PCT Publication No. WO 05/028443), lapatinib or lapatinib ditosylate (sold under the trademark Tykerb® by Glaxo SmithKline.

HER3 inhibitors: The HER3 antibodies or fragments thereof can be used with HER3 inhibitors which include, but are not limited to, MM-121, MM-111, IB4C3, 2DID12 (U3 Pharma AG), AMG888 (Amgen), AV-203 (Aveo), MEHD7945A (Genentech), and small molecules that inhibit HER3.

HER4 inhibitors: The HER3 antibodies or fragments thereof can be used with HER4 inhibitors.

PI3K inhibitors: The HER3 antibodies or fragments thereof can be used with PI3 kinase inhibitors which include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), BKM120 and BYL719.

mTOR inhibitors: The HER3 antibodies or fragments thereof can be used with mTOR inhibitors which include, but are not limited to, Temsirolimus (sold under the tradename Torisel® by Pfizer), ridaforolimus (formally known as deferolimus, (IR,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3, 10, 14,20-pentaoxo-11,36-dioxa-4-azatricyclo[30.3.1.04,9] hexatriaconta-16,24,26,28-tetraen-12-yl]propyl]-2-methoxycyclohexyl dimethylphosphinate, also known as Deforolimus, AP23573 and MK8669 (Ariad Pharm.), and described in PCT Publication No. WO 03/064383), everolimus (RADOOI) (sold under the tradename Afinitor® by Novartis), One or more therapeutic agents may be administered either simultaneously or before or after administration of a HER3 antibody or fragment thereof of the present invention.

### Methods of Producing Antibodies of the Invention

### Nucleic Acids Encoding the Antibodies

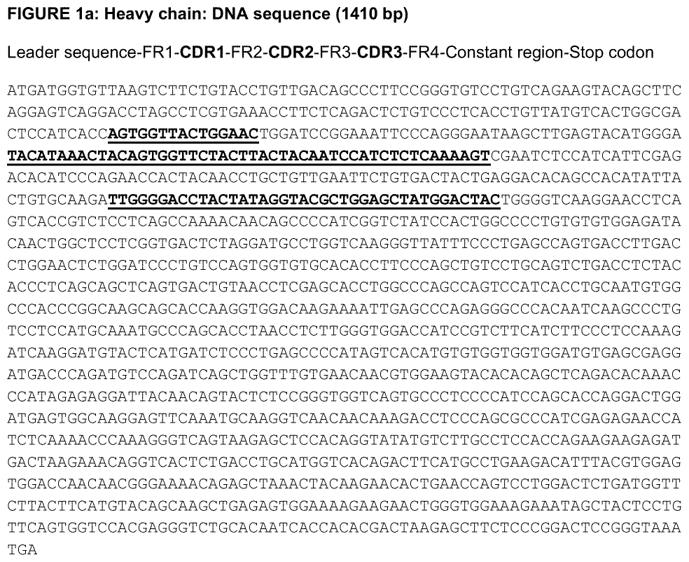

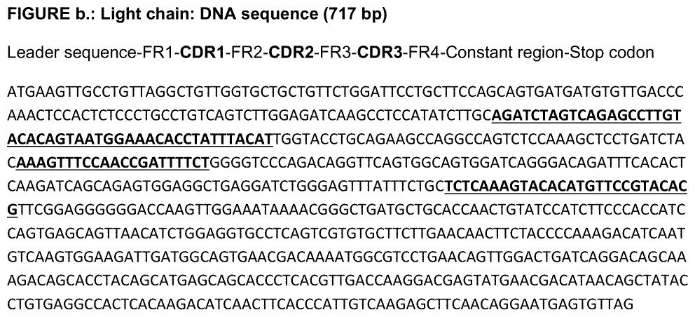

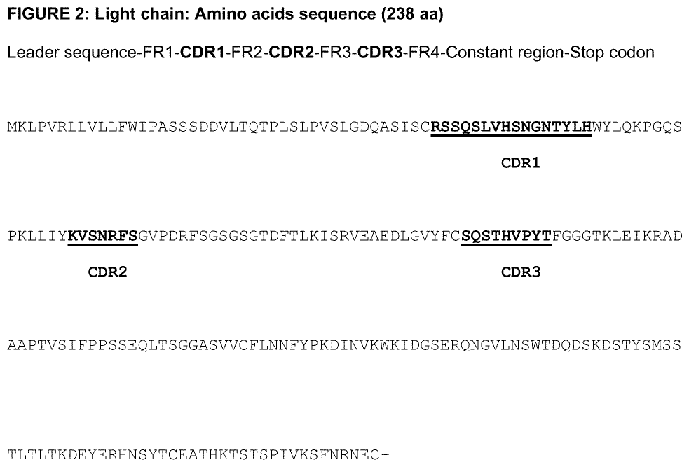

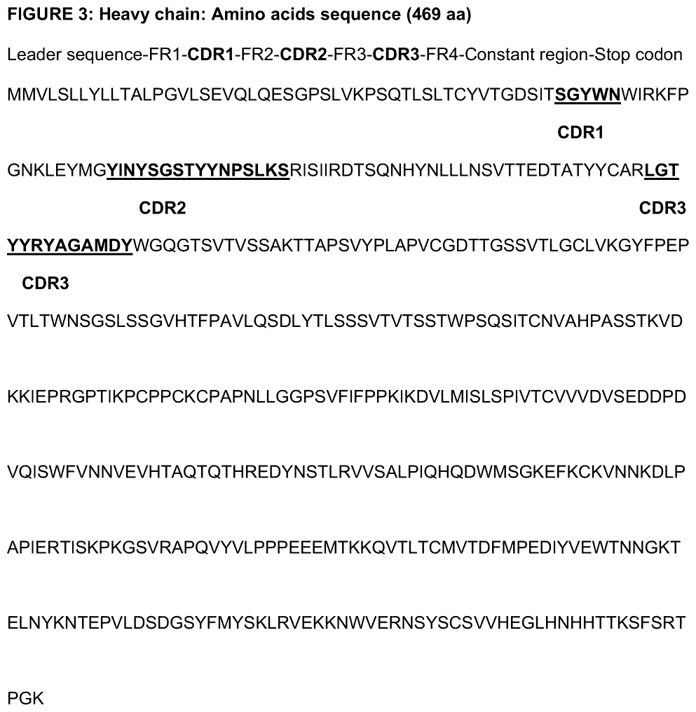

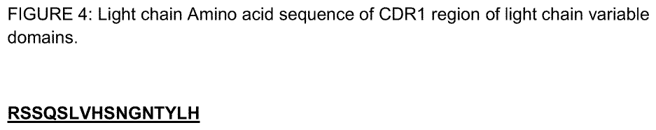

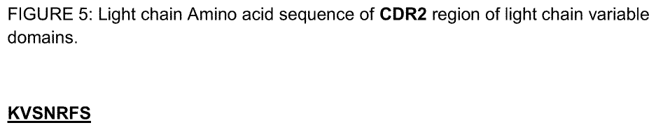

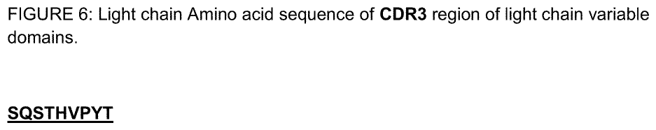

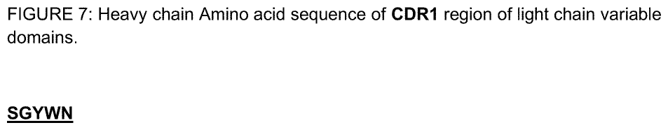

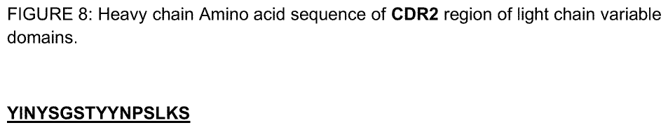

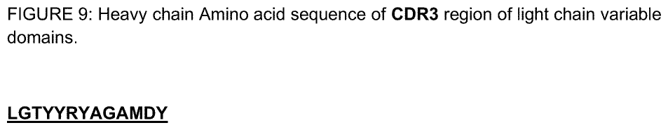

The invention provides substantially purified nucleic acid molecules which encode polypeptides comprising segments or domains of the HER3 antibody chains described above. Some of the nucleic acids of the invention comprise the nucleotide sequence encoding the HER3 antibody heavy chain variable region, and/or the nucleotide sequence encoding the light chain variable region. In a specific embodiment, the nucleic acid molecules are those identified in FIG. 1 (A and B).

Also provided in the invention are polynucleotides which encode at least one CDR region and usually all three CDR regions from the heavy or light chain of the antibody or fragment thereof set forth FIG. 1 (A and B). Some other polynucleotides encode all or substantially all of the variable region sequence of the heavy chain and/or the light chain of the antibody or fragment thereof set forth above. Because of the degeneracy of the code, a variety of nucleic acid sequences will encode each of the immunoglobulin amino acid sequences.

Also provided in the invention are expression vectors and host cells for producing the antibodies or fragments thereof. Various expression vectors can be employed to express the polynucleotides encoding the HER3 antibody chains or fragments thereof. Both viral-based and nonviral expression vectors can be used to produce the antibodies in a mammalian host cell. Nonviral vectors and systems include plasm ids, episomal vectors, typically with an expression cassette for expressing a protein or RNA, and human artificial chromosomes (see, e.g., Harrington et al, (1997) Nat Genet 15:345). For example, nonviral vectors useful for expression of the HER3 polynucleotides and polypeptides in mammalian {e.g. human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus. See, Brent et al, (1995); Smith, Annu. Rev. Microbiol. 49:807; and Rosenfeld et al, (1992) Cell 68:143.

The host cells for harboring and expressing the antibody or fragment chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). Other microbes, such as yeast, can also be employed to express antibodies or fragments thereof. Insect cells in combination with baculovirus vectors can also be used.

In some preferred embodiments, mammalian host cells are used to express and produce the antibodies or fragments thereof. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al, (1986) Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (Sambrook, et al). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, (1997) Cell 88:223), agent-enhanced uptake of DNA, and ex vivo transduction. For long-term, high-yield production of recombinant proteins, stable expression will often be desired. For example, cell lines which stably express antibody chains or fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Generation of Monoclonal Antibodies of the Invention

Monoclonal antibodies (mAbs) can be produced by a variety of techniques, including conventional monoclonal antibody methodology e.g., the standard somatic cell hybridization technique of Kohler and Milstein, (1975) Nature 256:495. Many techniques for producing monoclonal antibody can be employed e.g., viral or oncogenic transformation of B lymphocytes.

An animal system for preparing hybridomas is the murine system. Hybridoma production in the mouse is a well-established procedure. Immunization protocols and techniques for isolation of immunized splenocytes for fusion are known in the art. Fusion partners {e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine {e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

Human monoclonal antibodies directed against HER3 can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

Human monoclonal antibodies can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.

This invention also relates to processes for preparing an isolated HER3 binding protein, comprising the step of preparing the protein from a host cell that expresses the protein. Host cells that can be used include, without limitation, hybridomas, eukaryotic cells (e.g., mammalian cells such as hamster, rabbit, rat, pig, or mouse cells), plant cells, fungal cells, yeast cells {e.g., Saccharomyces cerevisiae or Pichia pastoris cells), prokaryotic cells {e.g., E. coli cells), and other cells used for production of binding proteins. Various methods for preparing and isolating binding proteins, such as scaffold proteins or antibodies, from host cells are known in the art and may be used in performing the methods described herein. Moreover, methods for preparing binding protein fragments, e.g., scaffold protein fragments or antibody fragments, such as papain or pepsin digestion, modern cloning techniques, techniques for preparing single chain antibody molecules and diabodies also are known to those skilled in the art and may be used in performing the presently described methods.

### Characterization of the Antibodies of the Invention

The antibodies of the invention can be characterized by various functional assays. For example, they can be characterized by their ability to inhibit biological activity by inhibiting HER signaling in a phospho-HER assay, their affinity to a HER3 protein {e.g., human HER3), the epitope binning, their resistance to proteolysis, and their ability to block HER3 downstream signaling. Various methods can be used to measure HER3-mediated signaling. For example, the HER signaling pathway can be monitored by (i) measurement of phospho-HER3; (ii) measurement of phosphorylation of HER3 or other downstream signaling proteins (e.g. Akt), (iii) ligand blocking assays (iv) heterodimer formation, (v) HER3 dependent gene expression signature, (vi) receptor internalization, and (vii) HER3 driven cell phenotypes (e.g. proliferation).

The ability of an antibody to bind to HER3 can be detected by labelling the antibody of interest directly, or the antibody may be unlabeled and binding detected indirectly using various sandwich assay formats known in the art.

To demonstrate binding of monoclonal HER3 antibodies to live cells expressing a HER3 protein, flow cytometry can be used. Briefly, cell lines expressing HER3 can be mixed with various concentrations of a HER3-binding antibody in PBS containing 0.1% BSA and 10% fetal calf serum, and incubated at 4° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells.

The antibodies or fragments thereof of the invention can be further tested for reactivity with a HER3 polypeptide or antigenic fragment by Western blotting. Briefly, purified HER3 polypeptides or fusion proteins, or cell extracts from cells expressing HER3 can be prepared and subjected to sodium dodecyl sulfate polyacrylamide gel electrophoresis. After electrophoresis, the separated antigens are transferred to nitrocellulose membranes, blocked with 10% BSA, and probed with the monoclonal antibodies of the invention. IgG binding can be detected using anti-IgG alkaline phosphatase and developed with ECL plus detection system.

A number of readouts can be used to assess the efficacy, and specificity, of HER3 antibodies in cell-based assays of ligand-induced heterodimer formation. Activity can be assessed by one or more of the following:

Functional activity can also be assessed by Inhibition of the activation of signaling pathways by ligand-activated heterodimerization. Association with HER3 appears a key for other members of the EGF family of receptors to elicit maximal cellular response following ligand binding. In the case of the kinase-defective HER3, HER2 provides a functional tyrosine kinase domain to enable signaling to occur following binding of growth factor ligands. Thus, cells co-expressing HER2 and HER3 can be treated with ligand, for example heregulin, in the absence and presence of inhibitor and the effect on HER3 tyrosine phosphorylation monitored by a number of ways including immunoprecipitation of HER3 from treated cell lysates and subsequent Western blotting using anti-phosphotyrosine antibodies. Alternatively, a high-throughput assay can be developed by trapping HER3 from solubilized lysates onto the wells of a 96-well plate coated with an anti-HER3 receptor antibody, and the level of tyrosine phosphorylation measured using, for example, europium-labelled anti-phosphotyrosine antibodies, as embodied by Waddleton et ah, (2002) Anal. Biochem. 309: 150-157.

### Inhibition of Cellular Proliferation.

A variety of cell lines are known to co-express combinations of ErbB receptors, for example many breast and prostate cancer cell lines. Assays may be performed in 24/48/96-well formats with the readout based around DNA synthesis (tritiated thymidine incorporation), increase in cell number or tumor colonies (crystal violet staining or Cyquant proliferation assay or CellTiterGlo assay).

Ability of antibodies or fragments thereof to block in vivo growth of tumour xenografts of human tumour cell lines whose tumorigenic phenotype is known to be at least partly dependent on ligand activation of HER3 heterodimer cell signaling e.g. BxPC3 pancreatic cancer cells or SK-BR-3: human breast cancer cells etc. This can be assessed in immunocompromised mice either alone or in combination with an appropriate cytotoxic agent for the cell line in question. Examples of functional assays are also described in the Example section below.

### Prophylactic and Therapeutic Uses

The present invention provides methods of treating a disease or disorder associated with the HER3 signaling pathway by administering to a subject in need thereof an effective amount of the antibody or fragment thereof of the invention. In a specific embodiment, the present invention provides a method of treating or preventing cancers (e.g., breast cancer, colorectal cancer, lung cancer, multiple myeloma, ovarian cancer, liver cancer, gastric cancer, pancreatic cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, melanoma, prostate cancer, benign prostatic hyperplasia, gynacomastica, and endometriosis) by administering to a subject in need thereof an effective amount of the antibodies or fragments thereof of the invention. In some embodiments, the present invention provides methods of treating or preventing cancers associated with a HER3 signaling pathway by administering to a subject in need thereof an effective amount of the antibodies of the invention.

The antibodies or fragments thereof of the invention can also be used to treat or prevent other disorders associated with aberrant or defective HER3 signaling, including but are not limited to respiratory diseases, osteoporosis, osteoarthritis, polycystic kidney disease, diabetes, schizophrenia, vascular disease, cardiac disease, non-oncogenic proliferative diseases, fibrosis, and neurodegenerative diseases such as Alzheimer's disease.

Suitable agents for combination treatment with HER3 antibodies include standard of care agents known in the art that are able to modulate the ErbB signaling pathway. Suitable examples of standard of care agents for HER2 include, but are not limited to Herceptin and Tykerb. Suitable examples of standard of care agents for EGFR include, but are not limited to Iressa, Tarceva, Erbitux and Vectibix. Other agents that may be suitable for combination treatment with HER3 antibodies include, but are not limited to those that modulate receptor tyrosine kinases, G-protein coupled receptors, growth/survival signal transduction pathways, nuclear hormone receptors, apoptotic pathways, cell cycle and angiogenesis.

### Detection/Theranostics and Diagnostic Uses

In another aspect, the invention features methods for detecting the presence of HER3 in a sample, e.g., in vitro or in vivo (e.g., a biological sample, e.g., serum, semen or urine, or a tissue biopsy, e.g., from a hyperproliferative or cancerous lesion). The subject method can be used to evaluate (e.g., monitor treatment or progression of, diagnose and/or stage a disorder described herein, e.g., a hyperproliferative or cancerous disorder, in a subject). The method includes: (i) contacting the sample with (and optionally, a reference, e.g., a control sample), or administering to the subject, an antibody molecule as described herein, under conditions that allow interaction to occur, and (ii) detecting formation of a complex between the antibody molecule, and the sample (and optionally, the reference, e.g., control, sample). Formation of the complex is indicative of the presence of HER3, and can indicate the suitability or need for a treatment described herein. In some embodiments, HER3 is detected prior to treatment, e.g., prior to an initial treatment, or prior to a treatment after a treatment interval. Detection can involve an immunohistochemistry, immunocytochemistry, FACS, antibody molecule complexed magnetic beads, ELISA assays, PCR-techniques (e.g., RT-PCR), or an in vivo imaging technique. Typically, the antibody molecule used in the in vivo and in vitro detection methods is directly or indirectly labeled with a detectable substance to facilitate detection of the bound or unbound binding agent. Suitable detectable substances include various biologically active enzymes, prosthetic groups, fluorescent materials, luminescent materials, paramagnetic (e.g., nuclear magnetic resonance active) materials, and radioactive materials. In other embodiments, the antibody molecule is detected in vivo, e.g., using an in vivo imaging technique as described herein (e.g., PET imaging).

Additional embodiments provide a method of treating a cancer, comprising: testing a subject, e.g., a sample (e.g., a subject's sample comprising cancer cells) for the presence of HER3 and also of one, two or all of NRG1-rearranged fusions: (Cluster of Differentiation 74-Neuregulin-1) CD74-NRG1 fusion, (Solute Carrier Family 3 Member 2-Neuregulin-1) SLC3A2-NRG1 fusion, (Syndecan-4-Neuregulin-1) SDC4-NRG1 fusion, DOC4-NRG1 fusion, (Rho-associated protein kinase 1-Neuregulin-1) ROCK1-NRG1 fusion, (Forkhead Box A1-Neuregulin-1) FOXA1-NRG1 fusion, (A-Kinase Anchoring Protein 13-Neuregulin-1) AKAP13-NRG1 fusion, (Thrombospondin 1-Neuregulin-1) THBS1-NRG1 fusion, (Phosphodiesterase 7A-Neuregulin-1) PDE7A-NRG1 fusion, (ATPase Na+/K+ Transporting Subunit Beta 1-Neuregulin-1) ATP1B1-NRG1 fusion, NRG1-PMEPA1 fusion, Clusterin-NRG1 fusion. Administering a therapeutically effective amount of the anti-HER3 antibody described herein to the subject, optionally in combination with one or more other agents, thereby treating the cancer.

In one aspect, the invention encompasses diagnostic assays for determining HER3 and/or nucleic acid expression as well as HER3 protein function, in the context of a biological sample (e.g., blood, serum, cells, tissues) or from individual afflicted with cancer, or is at risk of developing cancer.

### Pharmaceutical Compositions

To prepare pharmaceutical or sterile compositions including antibodies or fragments thereof, the antibodies or fragments thereof are mixed with a pharmaceutically acceptable carrier or excipient. The compositions can additionally contain one or more other therapeutic agents that are suitable for treating or preventing cancer.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions.

For antibodies or fragments thereof of the invention, the dosage administered to a patient may be 0.0001 mg/kg to 100 mg/kg of the patient's body weight.

For antibody thereof, the dosage administered to a patient is typically 0.1 mg/kg to 100 mg/kg of the patient's body weight. Preferably, the dosage administered to a patient is between 0.1 mg/kg and 20 mg/kg of the patient body weight, more preferably 1 mg/kg to 20 mg/kg of the patient's body weight. The dosage and frequency of administration of Antibody thereof, can be reduced by enhancing uptake and tissue penetration of the antibody or antigen binding portion thereof, by modifications such as, for example, lipidation.

A composition of the present invention may also be administered via one or more routes of administration using one or more of a variety of methods known in the art. As will be appreciated by the skilled artisan, the route and/or mode of administration will vary depending upon the desired results. Selected routes of administration for antibodies or fragments thereof of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically.

In some embodiments, antibody thereof, can be administered alone or in combination with other types of treatments (e.g., radiation therapy, chemotherapy, hormonal therapy, therapeutic antibodies, immunotherapy and anti-tumor agents).

In some embodiments, a method is provided for diagnosing a disease (e.g., a cancer) associated with HER3 upregulation in a subject, by contacting antibody disclosed herein (e.g., ex vivo or in vivo) with cells from the subject, and measuring the level of binding to HER3 on the cells. Abnormally high levels of binding to HER3 indicate that the subject has a disease associated with HER3 upregulation.

In some embodiments, a method for suppressing tumor growth is provided. The method can include providing an HER3 antibody to a tumor that comprises a cell that expresses HER3, thereby suppressing tumor growth.

In some embodiments, the antibody disclosed herein can be used to inhibit, block, and/or reduce the proliferation of various cells in vitro, in vivo, or ex vivo. In some embodiments, the antibody can block or reduce the proliferation of various cells (e.g., epithelial, colorectal, and/or pancreatic cancer cell lines) in the absence of NRG.

In some embodiments, the antibody thereof, inhibit proliferation of cells in the presence and/or absence of an HER3 activator, such as NRG-1. In some embodiments, the antibody can achieve 5, 10, 15, 20, 25, 30, 35, 40, 45, 50% or greater inhibition of HER3 signaling in the absence of NRG-1. In some embodiments, the antibody can achieve 5, 10, 15, 20, 25, 30, 35, 40, 45, 50% or greater inhibition of HER3 signaling in the presence of NRG-1. In some embodiments, the Antibody thereof, can prevent and/or reduce NRG-1 driven signaling, even when NRG is already bound to HER3.

In some embodiments, the antibody thereof, block one or more of the functions or activities of HER3 disclosed herein. In some embodiments, the Antibody thereof, reduce and/or block NRG binding to HER3. In some embodiments, the Antibody thereof, block or reduce dimerization of HER3 with another HER3 molecule and/or EGFR, and/or HER2, and/or ErbB4.

In some embodiments, the antibody or antigen binding portions thereof, can be used to reduce a cancer's resistance, or increase the sensitivity, to another therapy.

In some embodiments, one or more of the antibody thereof, noted herein can enhance the antiproliferative activity of ErbB targeted antibodies. In some embodiments, the Antibody thereof, can be combined with cetuximab or panitimumab to provide a composition with enhanced antiproliferative activity. In some embodiments, the antibody thereof, disclosed herein can be combined with anti-HER2 antibody or anti-HER2 antibodies such as trastuzumab and/or pertuzumab. In some embodiments, any one or more of the sur-binding proteins provided herein can be combined with other molecules. In some embodiments, this can provide for enhanced effectiveness. In some embodiments, the antibody or antigen binding portions thereof, can be combined with cetuximab, panitimumab, pertuzumab, trastuzumab, lapatinib, GDC-0941 to provide a composition with enhanced antiproliferative activity and/or improved inhibition. In some embodiments, this can be effective in the presence of NRG. In some embodiments, this can be effective in the absence of NRG. In some embodiments, this allows for a greater amount of inhibition to be achieved than either molecule acting alone. In some embodiments, antibody can be combined with at least one of cetuximab, panitimumab, pertuzumab, trastuzumab to provide for at least one of the following: reduction in cell surface HER3, enhancement in antiproliferative activity for EGFR targeted molecules (antibodies or other molecules), enhance the antiproliferative activity of ERB2 targeted molecules (antibodies or other molecules), enhance the activity of PI3K, AKT, mTOR targeted molecules, reduction in ligand-induced HER3 phosphorylation, AKT phosphorylation, and/or ERK phosphorylation, and/or improvement in the inhibition of proliferation of cancer cell line (including any provided herein, such as breast cancer cells). In some embodiments, the amount of any of the second or third therapeutic antibody provided herein can be used at an amount of at least 0.001 mg/kg of subject weight, e.g., 0.001, 0.01, 0.1, 1, 10, 20, 30, 40, 50, 60, 70, 80, 90, or 100 mg/kg of subject weight, including any range defined between any two of the preceding values. In some embodiments, the amount of the sur-binding protein used is from 0.1 to 100 mg/kg.

In some embodiments, in the combination of any of the antibodies noted herein and any of the proteins, the amount of any of the antibodies provided herein can be used in an amount of at least 0.001 mg/kg of subject weight, e.g., 0.001, 0.01, 0.1, 1, 10, 20, 30, 40, 50, 60, 70, 80, 90, or 100 mg/kg of subject weight, including any range defined between any two of the preceding values. In some embodiments, the amount of the antibody used is from 0.1 to 100 mg/kg. In some embodiments, more of the antibody of the present invention is used than a second suitable antibody such as Anti-EGFR or Anti-HER2.

In some embodiments, the antibody can reduce tumor growth in vivo in both HER2-overexpressing and non-overexpressing cells. In some embodiments, the antibody is at least as effective as at least one or more of: cetuximab, panitimumab, pertuzumab, trastuzumab. In some embodiments, the antibody is at least as effective as one or more of cetuximab, panitimumab, pertuzumab, trastuzumab, Ab B, or Ab A and the antibody is more potent. In some embodiments, the antibody is at least 1% more potent, e.g., 1, 5, 10, 20, 30, 40, 50, 60, 70, 80, 90, 100, 200, 300, 500, 1000, 5000, 10,000, 100,000, 1,000,000, or 10,000,000 percent more potent, including any range of potencies defined between any two of the preceding values. In some embodiments, the antibody acts to reduce cell proliferation in a manner that is not limited to a NRG-stimulated growth mechanism. In some embodiments, the antibody can work via a mechanism of action that is distinct from other ErbB approaches (e.g., independent of NRG), and still maintain an ability to augment EGFR inhibitors (e.g., any of the inhibitors provided herein). In some embodiments, the antibody decreases cell surface HER3. In some embodiments, the antibody decreases cell surface expression of HER3.

In some embodiments, any one or more of the Antibody can augment another (non-antibody and/or non-HER3) drug that inhibits EGFR. In some embodiments, the antibody are administered or included in a composition without another active ingredient (and/or without a different HER3 inhibitor).

In some embodiments, the antibody thereof, are effective against cells that are resistant to EGFR antibodies. In some embodiments, the Antibody thereof, are effective against cells that are resistant to inhibitors of EGFR tyrosine kinase activity. In some embodiments, the Antibody thereof, are effective against cells bearing K-ras gene variants. In some embodiments, the antibody thereof, is effective against lung cancer. In some embodiments, the antibody thereof, is effective for a subject having lung cancer and a mutation in a K-ras gene. In some embodiments, the antibody thereof, is effective for a subject having pancreatic cancer and a mutation in a K-ras gene. In some embodiments, one first tests a subject for the presence or absence of a K-ras gene variation. In situations where the subject has a K-ras point mutation, the subject is administered antibody thereof, as disclosed herein.

In some embodiments, the antibody can prolong survival in a mouse model for at least some percentage of a population of mice out past 60 days. In some embodiments, the antibody can extend survival to more than 10% of a mouse population to, and/or beyond, 70, 75, or 80 days. In some embodiments, the antibody can extend survival to more than 20% of a mouse population to, and/or beyond, 70, 75, or 80 days. In some embodiments, the antibody can extend survival to about 30% of a mouse population to, and/or beyond, 70, 75, or 80 days.

Various delivery systems are known and can be used to administer a antibody, e.g., encapsulation in liposomes, microparticles, microcapsules, recombinant cells capable of expressing the antibody thereof, receptor-mediated endocytosis (see, e.g., Wu and Wu, J. Biol. Chem. 262:4429-4432, 1987), construction of a nucleic acid as part of a retroviral or other vector, etc. Methods of introduction include but are not limited to intradermal, intramuscular, intraperitoneal, intravenous, subcutaneous, intranasal, epidural, and oral routes. The antibody thereof, can be administered by any convenient route, for example by infusion or bolus injection, by absorption through epithelial or mucocutaneous linings (e.g., oral mucosa, rectal and intestinal mucosa, etc.), and can be administered together with other biologically active agents. Administration can be systemic or local. In addition, it can be desirable to introduce the antibody or antigen binding portion thereof into the central nervous system by any suitable route, including intraventricular and intrathecal injection; intraventricular injection can be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In some embodiments, it may be desirable to administer the antibody thereof, and/or antibodies locally to the area in need of treatment; this can be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In some embodiments, when administering antibody or antigen binding portion thereof, care can be taken to use materials to which the antibody or antigen binding portion thereof, does not absorb.

In some embodiments, the antibody or antigen binding fragment thereof or antibody construct disclosed herein may also be formulated as immunoliposomes. A “liposome” is a small vesicle composed of various types of lipids, phospholipids and/or surfactant which is useful for delivery of a drug to a mammal. Fab′ fragments of the antibody of the present invention can be conjugated to the liposomes as described in Martin et al. J. Biol. Chem. 257: 286-288 (1982) via a disulfide interchange reaction. A chemotherapeutic agent is optionally contained within the liposome.

Antibody, antigen binding portions thereof, and/or antibodies can also be provided in a pharmaceutical composition. Such compositions can comprise a therapeutically effective amount of antibody, antigen binding portion thereof, and/or antibody and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

In another aspect, the present invention provides a composition, e.g., a pharmaceutical composition, containing one or a combination of an Antibody, antigen binding portions thereof, and/or antibodies thereof disclosed herein, formulated together with a pharmaceutically acceptable carrier. In some embodiments, the compositions include a combination of multiple (e.g., two or more) isolated agents, which bind different epitopes on HER3.

For the therapeutic compositions, formulations of the present disclosure include those suitable for oral, nasal, topical (including buccal and sublingual), transdermal, subcutaneous, intrathecal, intraspinal, rectal, vaginal and/or parenteral administration. The formulations can conveniently be presented in unit dosage form and can be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

HER binding agents can further encompass any pharmaceutically acceptable salts, esters, or salts of such esters, or any other compound which, upon administration to an animal including a human, is capable of providing (directly or indirectly) the biologically active metabolite or residue thereof.

In some embodiments, the composition comprises at least one antibody or antigen binding portion thereof, that binds HER3 and a second antibody or antigen binding portion thereof, that binds EGFR. In some embodiments, the composition comprises at least one antibody or antigen binding portion thereof, that binds HER3 and a second antibody or antigen binding portion thereof, that binds HER2. In some embodiments, the composition comprises at least one antibody or antigen binding portion thereof, that binds HER3 and a second antibody or antigen binding portion thereof, that binds ErbB4. In some embodiments, the composition comprises at least one antibody or antigen binding portion thereof, that binds HER3 and an antibody that binds that ErbB 1. In some embodiments, the composition comprises at least one antibody or antigen binding portion thereof, that binds HER3 and an antibody that binds HER2. In some embodiments, the composition comprises at least one antibody or antigen binding portion thereof, that binds HER3 and an antibody that binds HER3. In some embodiments, the composition comprises at least one antibody or antigen binding portion thereof, that binds HER3 and an antibody that binds ErbB4. In some embodiments, the composition comprises at least one antibody that binds HER3 and an antibody that binds HER2. For these aforementioned embodiments, the Antibody can either be a single bispecific construct or a pair of constructs

In some embodiments, one can combine antibody or antigen binding portion thereof that binds HER3 and a second antibody or antigen binding portion thereof, that binds, with one or more growth factors, non-ErbB receptors, and/or immune cell recruitment specificities to increase tumor cell killing. For these embodiments, the Antibody can either be a single bispecific construct or a pair of constructs.

In some embodiments, a antibody or antigen binding portion thereof, can be combined with one or more traditional chemotherapeutic, growth factor tyrosine kinase inhibitor, protein kinase inhibitor, caspase or apoptotic activators, microtubule inhibitors (e.g. taxanes), estrogen receptor inhibitors (tamoxifin), and/or aromatase inhibitors, HSP90 inhibitors.

In some embodiments, a method for suppressing a cancerous cell is provided. The method can comprise identifying a subject having a cancerous cell, wherein the cancerous cell expresses HER3, and administering to the subject an HER3 sur-binding protein in an amount sufficient to bind to HER3 on the cancerous cell and thereby block the Ras/Raf/MEK pathway.

In some embodiments, a method for suppressing a cancerous cell is provided. The method can comprise identifying a subject having a cancerous cell, wherein said cancerous cell expresses HER3, and administering to the subject an HER3 sur-binding protein in an amount sufficient to bind to HER3 on the cancerous cell and thereby block the PI3K, AKT, or PI3K and AKT pathway.

Some breast cancer patients are unresponsive to anti-HER2 treatment, such as trastuzumab. One mechanism for trastuzumab resistance in HER2-positive breast cancer involves truncation of the HER2 such that the extracellular domain to which trastuzumab binds is absent. HER2 truncation can occur by several mechanisms including proteolytic shedding and alternative initiation of translation using internal methionine residues that exclude trastuzumab and other epitopes. In either of these cases expression of truncated HER2 (“p95 HER-2”) has been shown to be a negative prognostic factor and defines a group of patients with significantly worse outcome.

As outlined in the examples below, in some embodiments, one can use anti-HER3 antibody and/or sur-binding protein to treat anti-HER2 unresponsive tumors, such as those that have truncated HER2. These tumors are expected to be resistant to both trastuzumab and pertuzumab therapy. To demonstrate these benefits experimentally, one can use a human tumor cell line bearing HER3 and truncated HER2 and test the HER3 Sur-binding proteins for inhibition of proliferation or HER3 mediated signaling, similar to in vitro assays described previously. Alternatively, cultured tumor cells that bear HER3, can be transiently or stably transfected or transduced to overexpress truncated HER2. Different deleted forms of Her-2 could be introduced to recapitulate proteolytically cleaved or the alternatively translated forms and the resulting cell lines or pools could be tested to demonstrate their responsiveness to anti-HER3. Since the binding of anti-HER3 sur-binding proteins is independent of the presence of HER2, and since they inhibit the growth of HER2 driven tumors, they are expected inhibit growth of tumors expressing truncated HER2 and benefit this patient population.

As the binding of anti-HER3 sur-binding proteins is independent of the presence of HER2, and since they inhibit the growth of HER2 driven tumors, they are predicted to inhibit growth of tumors expressing truncated HER2 and benefit this patient population.

In some embodiments, the antibody provided herein can be used in combination with an MTOR (mammalian target of rapamycin) inhibitor. mTORC1 acts in a feedback pathway to reduce signaling through PI3K and mTORC2. Examples of mTOR inhibitors include, but are not limited to temsirolimus, everolimus, ridaforolimus and BEZ235. In some embodiments, the method can include identifying a subject at risk of developing a cancer, administering a dose of anti-HER3 antibody of the present invention either prior to, subsequent to, or in combination with one or more inhibitors of mTOR. The dose of the antibody can be varied, for example, an amount that is effective on its own or an amount that is effective in combination with the inhibitor of mTOR.

The pharmaceutical compositions provided herein can be especially useful for diagnosis, prevention, or treatment of a hyperproliferative disease. The hyperproliferative disease can be associated with increased HER family signal transduction. In particular, the disease can be associated with increased HER3 phosphorylation, increased complex formation between HER3 and other members of the HER family, increased PI3 kinase activity, increased c-jun terminal kinase activity and/or AKT activity, increased ERK2 and/or PYK2 activity, or any combination thereof. The hyperproliferative disease can be, for example, selected from the group consisting of breast cancer, gastrointestinal cancer, pancreatic cancer, prostate cancer, ovarian cancer, stomach cancer, endometrial cancer, salivary gland cancer, lung cancer, kidney cancer, colon cancer, colorectal cancer, thyroid cancer, bladder cancer, glioma, melanoma, or other HER3 expressing or overexpressing cancers, and the formation of tumor metastases.

In addition to the above, other embodiments of combination therapies of the invention include the following: for the treatment of breast cancer, a HER3 antibody or a therapeutic conjugate thereof, in combination with methotrexate, paclitaxel, doxorubicin, carboplatin, cyclophosphamide, daunorubicin, epirubicin, 5-fluorouracil, gemcitabine, ixabepilone, mutamycin, mitoxantrone, vinorelbine, docetaxel, thiotepa, vincristine, capecitabine, an EGFR antibody (e.g. zalutumumab, cetuximab, panitumumab or nimotuzumab) or other EGFR inhibitor (such as gefitinib or erlotinib), HER2 antibody or -conjugate (such as, e.g., trastuzumab, trastuzumab-DM1 or pertuzumab), an inhibitor of both EGFR and HER2 (such as lapatinib), and/or in combination with a HER3 inhibitor.

For the treatment of non-small-cell lung cancer, a HER3 antibody in combination with EGFR inhibitors, such as an EGFR antibody, e.g. zalutumumab, cetuximab, panitumumab or nimotuzumab or other EGFR inhibitors (such as gefitinib or erlotinib), or in combination with HER2 agent (such as a HER2 antibody, e.g. trastuzumab, trastuzumab-DM1 or pertuzumab) or in combination with an inhibitor of both EGFR and HER2, such as lapatinib, or in combination with a HER3 inhibitor.

For the treatment of colorectal cancer a HER3 antibody in combination with one or more compounds selected from: gemcitabine, bevacizumab, FOLFOX, FOLFIRI, XELOX, IFL, oxaliplatin, irinotecan, 5-FU/LV, Capecitabine, UFT, EGFR targeting agents, such as cetuximab, panitumumab, zalutumumab; VEGF inhibitors, or tyrosine kinase inhibitors such as sunitinib.

For the treatment of prostate cancer a HER3 antibody in combination with one or more compounds selected from: hormonal/antihormonal therapies; such as antiandrogens, Luteinizing hormone releasing hormone agonists, and chemotherapeutics such as taxanes, mitoxantrone, estramustine, 5FU, vinblastine, and ixabepilone.

### EXAMPLES

The invention having been fully described, it is further illustrated by the following examples and claims, which are illustrative and are not meant to be further limiting. It should be understood that this invention is not limited to the particular methodology, protocols, and reagents, etc., described herein and as such may vary. It is understood that modifications can be made in the procedures set forth without departing from the spirit of the invention. The terminology used herein is for the purpose of describing particular embodiments only, and is not intended to limit the scope of the disclosed, which is defined solely by the claims.

### Example 1: Expression and Purification of Human Her3_aa20-643 from E. coli System

Human Her3_aa20-643 protein sequence:
MSEVGNSQAVCPGTLNGLSVTGDAENQYQTLYKLYERCEVVMGNLEIVLT
GHNADLSFLQWIREVTGYVLVAMNEFSTLPLPNLRVVRGTQVYDGKFAIF
VMLNYNTNSSHALRQLRLTQLTEILSGGVYIEKNDKLCHMDTIDWRDIVR
DRDAEIVVKDNGRSCPPCHEVCKGRCWGPGSEDCQTLTKTICAPQCNGHC
FGPNPNQCCHDECAGGCSGPQDTDCFACRHFNDSGACVPRCPQPLVYNKL
TFQLEPNPHTKYQYGGVCVASCPHNFVVDQTSCVRACPPDKMEVDKNGLK
MCEPCGGLCPKACEGTGSGSRFQTVDSSNIDGFVNCTKILGNLDFLITGL
NGDPWHKIPALDPEKLNVFRTVREITGYLNIQSWPPHMHNFSVFSNLTTI
GGRSLYNRGFSLLIMKNLNVTSLGFRSLKEISAGRIYISANRQLCYHHSL
NVVTKVLRGPTEERLDIKHNRPRRDCVAEGKVCDPLCSSGGCWGPGPGQC
LSCRNYSRGGVCVTHCNFLNGEPREFAHEAECFSCHPECQPMEGTATCNG
SGSDTCAQCAHFRDGPHCVSSCPHGVLGAKGPIYKYPDVQNECRPCHENC
TQGCKGPELQDCLGQTLVLIGKTHLT
Cloning strategy for human Her3_aa20-643
Target protein + His tag + stop codon
MSEVGNSQAVCPGTLNGLSVTGDAENQYQTLYKLYERCEVVMGNLEIVLT
GHNADLSFLQWIREVTGYVLVAMNEFSTLPLPNLRVVRGTQVYDGKFAIF
VMLNYNTNSSHALRQLRLTQLTEILSGGVYIEKNDKLCHMDTIDWRDIVR
DRDAEIVVKDNGRSCPPCHEVCKGRCWGPGSEDCQTLTKTICAPQCNGHC
FGPNPNQCCHDECAGGCSGPQDTDCFACRHFNDSGACVPRCPQPLVYNKL
TFQLEPNPHTKYQYGGVCVASCPHNFVVDQTSCVRACPPDKMEVDKNGLK
MCEPCGGLCPKACEGTGSGSRFQTVDSSNIDGFVNCTKILGNLDFLITGL
NGDPWHKIPALDPEKLNVFRTVREITGYLNIQSWPPHMHNFSVFSNLTTI
GGRSLYNRGFSLLIMKNLNVTSLGFRSLKEISAGRIYISANRQLCYHHSL
NWTKVLRGPTEERLDIKHNRPRRDCVAEGKVCDPLCSSGGCWGPGPGQCL
SCRNYSRGGVCVTHCNFLNGEPREFAHEAECFSCHPECQPMEGTATCNGS
GSDTCAQCAHFRDGPHCVSSCPHGVLGAKGPIYKYPDVQNECRPCHENCT
QGCKGPELQDCLGQTLVLIGKTHLTHHHHHH**

Gene synthesis with codon optimization was performed using standard methods known in the art and then subcloning was conducted into expression vector pET30a. Expression level evaluation was done by SDS-PAGE and Western blotting analysis. To obtain sufficient amount of HER3 protein a scale up expression using optimal conditions was undertaken. Followed by cell harvest and lysis (Inclusion body; re-dissolve inclusion body, purify HER3 protein with Ni-resin and followed by refolding. Quality control was performed using SDS-PAGE, Western Blotting and Bradford assay.

### Example 2: Generation of mAbs Against the Extracellular Domain of HER3

The extracellular domain of HER3 (20-643 a.a) was produced and used for the immunization of mice. Animals exhibiting suitable titers were identified, and lymphocytes were obtained from draining lymph nodes and, if necessary, pooled for each cohort. Lymphocytes were dissociated from lymphoid tissue by grinding in a suitable medium (for example, Dulbecco's Modified Eagle Medium (DMEM); to release the cells from the tissues, and suspended in DMEM. B cells were selected and/or expanded using standard methods, and fused with suitable fusion partner using techniques that were known in the art.

After several days of culture, the hybridoma supernatants were collected and subjected to screening assays as detailed in the examples below, including confirmation of binding to human HER3 by ELISA as well as the ability to kill cell lines in secondary Bioassays. Hybridoma lines that were identified to have the binding and functional properties of interest were then further selected and subjected to standard cloning and subcloning techniques. Clonal lines were expanded in vitro, and the secreted monoclonal antibodies obtained for analysis and gene sequencing was performed. Full nucleotide sequencing of cDNAs encoding the heavy and light chains identified 29Z6 Ab which was isotyped and identified as IgG2a molecule with kappa chain.

### Example 3: Binding of Anti-HER3 Antibody 29Z6

Antigen binding of IgG 29Z6 was analyzed in ELISA using immobilized HER3-Fc fusion comprising the extracellular domain (aa 20-643) of human HER3. The HER3-Fc fusion protein was coated onto polystyrene microtiter plates at 1 μg/ml in PBS. Remaining binding sites were blocked with PBS, 2% skimmed milk (MPBS). Plates were then incubated with a serial dilution of IgG 29Z6 in MPBS. After washing, bound antibody was detected with an HRP-conjugated anti-mouse Fc antibody and TMB, H₂O₂ as substrate. IgG 29Z6 showed specific, concentration-dependent binding to HER3 with an EC50 value in the subnanomolar range (0.88 nM).

### Example 4: Cross-Reactivity of Anti-HER3 Antibody 29Z6

IgG 29Z6 was able to detect the denatured and reduced HER3-Fc fusion proteins in immunoblotting experiments. Furthermore, we analyzed binding to human and mouse HER3-Fc fusion proteins in ELISA. Binding to both HER3-Fc fusion proteins was detected demonstrating that IgG 29Z6 is cross-reactive with HER3, thus the epitope of IgG 29Z6 is conserved in these two species.

### Example 5: Binding Specificity of Anti-HER3 29Z6 to HER3 Receptor

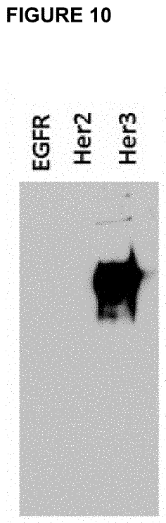

To demonstrate the binding specificity of 29Z6 antibody, ELISA assay was performed. ELISA plate was coated with 1 μg/ml of EGFR, HER2, or extracellular domain of HER3. The ELISA was incubated with increasing concentration of 29Z6 followed by incubation with HRP-goat-anti mouse IgG to detect 29Z6 antibody binding. Results showed 29Z6 bindings only to HER3. Confirmation of the binding specificity was undertaken using western blotting. Only HER3 was bound to 29Z6 while EGFR or HER2 didn't (FIG. 10).

### Example 6: Analysis of Binding Kinetics by Surface Plasmon Resonance

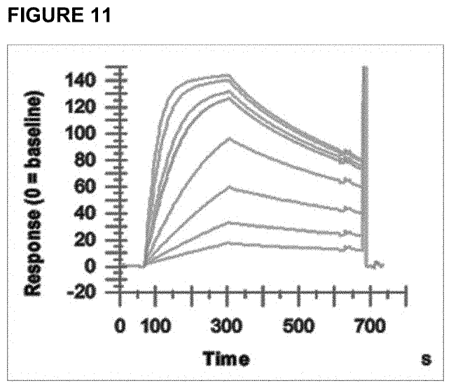

The binding kinetics of the 29Z6 mAb was analysed by surface plasmon resonance (SPR) using a Biacore3000 optical sensor platform equipped with research-grade CM5 sensor (GE Healthcare Bio-Sciences Corp., Piscataway, N.J.). Purified extracellular domain of HER3 was immobilized on the sensor chip surface of a carboxylated dextran-coated gold film using the standard amine coupling kit following the manufacturer's protocol. Briefly, 70 μL of a mixed solution of NHS/EDC (1:1, v/v) was injected to activate the carboxylated dextran, followed by manual injection of protein in 10 mM NaOAc (pH 4.5) until the desired surface density was reached. Ethanolamine 1 M in water (pH 8.5) was then injected to de-activate residual NHS-esters on the sensor chip. All binding experiments were carried out in HEPES buffer (10 mM HEPES, 150 mM NaCl, 3.4 mM EDTA, 0.005% Tween 20, pH 7.4) at 25° C. 29Z6 mAb at concentrations between 0.1 and 300 nM were injected randomly over the HER3 receptor ectodomain or antibody surfaces, respectively, at a flow rate of 20 μL·min-1 unless otherwise stated. After each injection, the surfaces were regenerated with two 30 s injections of 10 mM HCl. The resulting sensorgrams were aligned and double referenced using a mock activated surface and blank buffer injections. Kinetic data were evaluated by globally fitting the sensorgrams to a simple 1:1 interaction model using the Biacore software BiaEvaluation version 4.1 (GE Healthcare Bio-Sciences Corp.). The equilibrium KDs were determined from the resulting kinetic association and dissociation rates (kd/ka), and a minimum of nine independent runs were used to generate the reported standard deviations. A 1:1 binding model was used together with experimentally determined RMax as fixed parameter to determine association rate constant (ka), dissociation rate constant (kd), and equilibrium dissociation constant (KD). The resulting equilibrium dissociation constant (KD) was calculated to be 0.8±0.3×10⁻⁹ M while Ka 1.88×10⁵ [M⁻¹s⁻¹] and Kd 2.95×10⁻⁴ [s⁻¹] (FIG. 11).

### Example 7: Inhibition of Cell Proliferation by Anti-HER3 IgG 29Z6

SK-BR-3 (Mammary Gland Breast Adenocarcinoma cell line), FaDu (Hypopharyngeal Carcinoma cell line), BT-474 (Breast Ductal carcinoma cell line), PANC-1 (Pancreas Ductal Adenocarcinoma cell line) and MCF-7 (Breast Adenocarcinoma cell line), MDA-MB-231 (Human breast adenocarcinoma) cell lines were purchased from ATCC and routinely maintained in growth media supplemented with 10% fetal bovine serum (FBS). Human Lung Microvascular Endothelial Cells (HLMEC) and Human Umbilical Vein Endothelial Cells (HUVEC) were grown in specialized Endothelial Cell Growth Medium (Lonza).

SK-Br-3 cells were routinely cultured in McCoy's 5 A medium modified, supplemented with 10% fetal bovine serum and BT-474 cells were cultured in DMEM supplemented with 10% FBS. Sub-confluent cells were trypsinized, washed with PBS, diluted to 5×10⁴ cells/mL with growth media and plated in 96-well clear bottom black plates (Costar 3904) at a density of 5000 cells/well. The cells were incubated overnight at 37° C. before adding the appropriate concentration of HER3 antibody (typical final concentrations of 10 or 1 μg/mL). The plates were returned to the incubator for 6 days before assessing cell viability using Cyquant Cell Proliferation Assay (Thermofisher). The extent of growth inhibition obtained with each antibody was calculated by comparing the fluorescence values obtained with HER3 antibody to a standard isotype control antibody.

For proliferation assays FaDu (Hypopharyngeal Carcinoma cell line), BT-474 (Breast Ductal carcinoma cell line), PANC-1 (Pancreas Ductal Adenocarcinoma cell line), A549 (lung adenocarcinoma cell line), BxPC3 Luc (pancreas adenocarcinoma) and MCF-7 (Breast Adenocarcinoma cell line) cell lines were used. Cells were routinely cultured in DMEM/F12 (1:1) containing 4 mM L-Glutamine/15 mM HEPES/10% FBS. Sub-confluent cells were trypsinized, washed with PBS and diluted to 1×10⁵ cells/mL with DMEM/F12 (1:1) containing 4 mM L-Glutamine/15 mM HEPES/10 μg/mL Human Transferrin/0.2% BSA. Cells were plated in 96-well clear bottom black plates (Costar) at a density of 5000 cells/well. The appropriate concentration of HER3 antibody (typical final concentrations of 10 or 1 μg/mL) was then added. 40 ng/mL of NRGI-βI EGF domain (R&D Systems) was also added to the appropriate wells to stimulate cell growth. The plates were returned to the incubator for 6 days before assessing cell viability either using Cyquant Cell Proliferation Assay (Thermofisher) or CellTiterGlo assay (Promega). The extent of growth inhibition obtained with each antibody was calculated by comparing the fluorescence values obtained with HER3 antibody to a standard isotype control antibody.

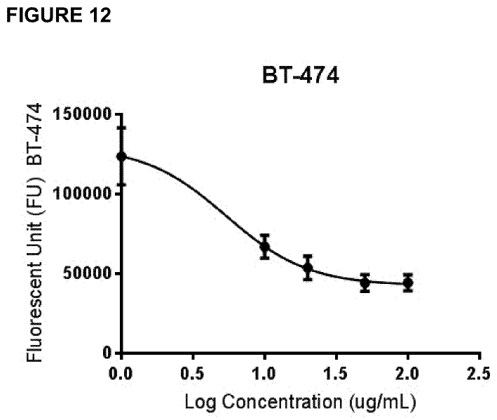

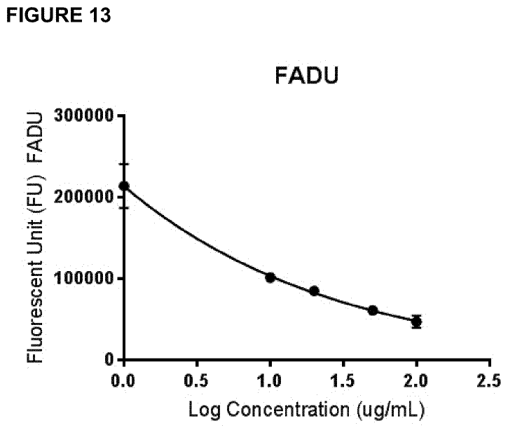

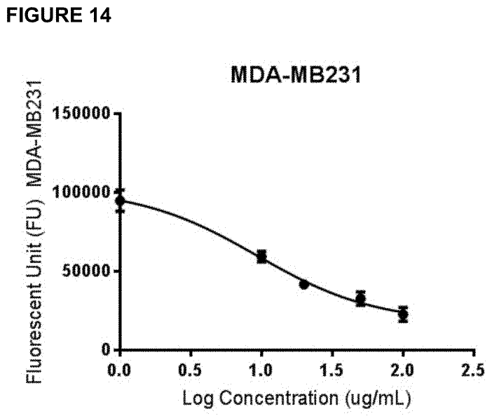

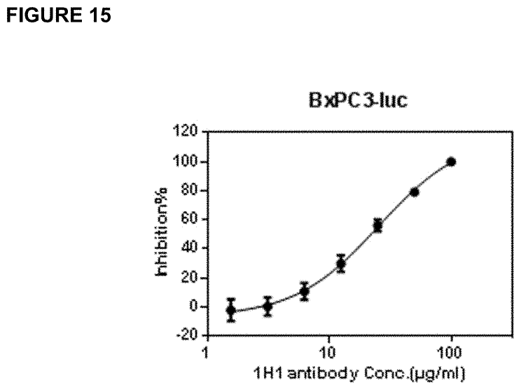

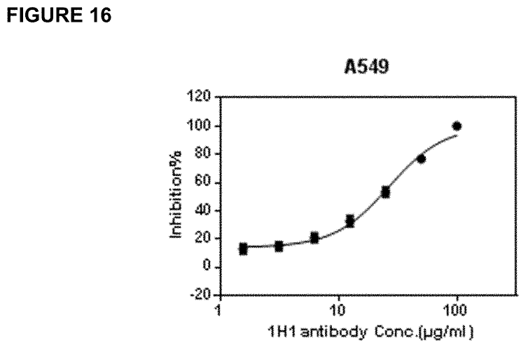

IgG 29Z6 was further evaluated concerning its ability to reduce tumor cell proliferation in vitro. To monitor this effect, various human cancer cell lines (MCF-7, BT-474, SKBR3, MDA-MB-231, PANC-1, FaDu, A549 and BxPC3) and were seeded at low density in 96 well plates, let adhere for one night, and were afterwards incubated under low serum concentration with IgG 29Z6 or IgG isotype as negative control. Proliferation was determined after 1 week of incubation. For all four cell lines a reduction on proliferation compared to control antibody was observed. For example, the IC50 in BT-474 cells was 5.3 μg/ml (FIG. 12) while in FaDu, an IC50 value of 3.3 μg/ml was determined under these conditions (FIG. 13). IC50 value was calculated to be 8.3 μg/ml in MDA-MB-231 cells (FIG. 14). IC50 value was calculated to be 18.1 μg/ml in BxPC3 Luc cells (FIG. 15) while IC50 value was calculated to be 20.3 μg/ml in A549 cells (FIG. 16). Normal human Lung Microvascular Endothelial Cells (HLMEC) and Human Umbilical Vein Endothelial Cells (HUVEC) were used as control and incubated with IgG 29Z6 to determine toxicity to normal cells. No toxicity was observed for human Lung Microvascular Endothelial Cells (HLMEC) and Human Umbilical Vein Endothelial Cells (HUVEC) with IgG 29Z6 concentration up to 100 μg/ml.

### Example 8: Inhibition of Colony Formation of Tumor Cells Incubated with IgG 29Z6

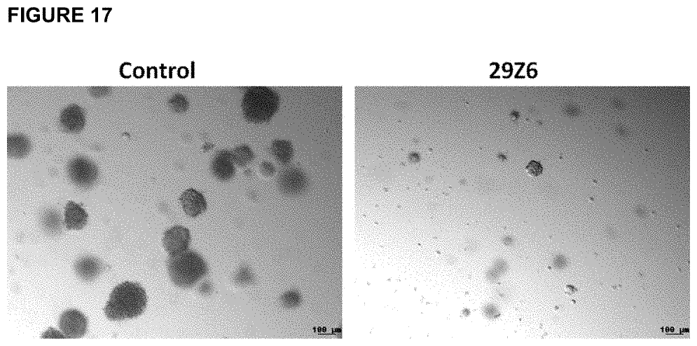

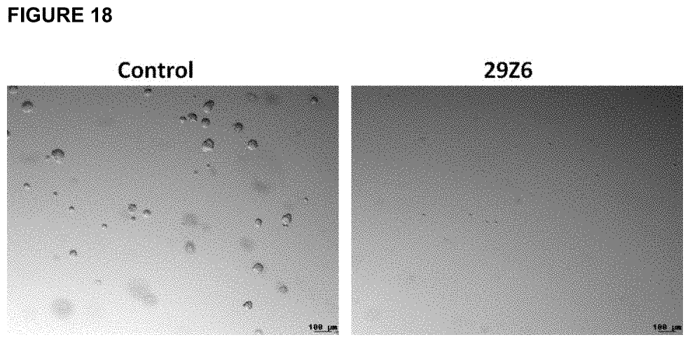

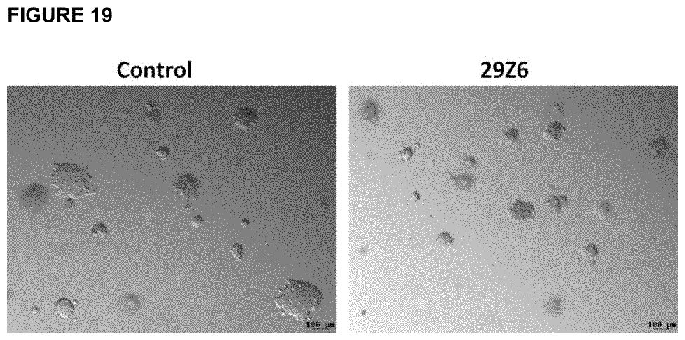

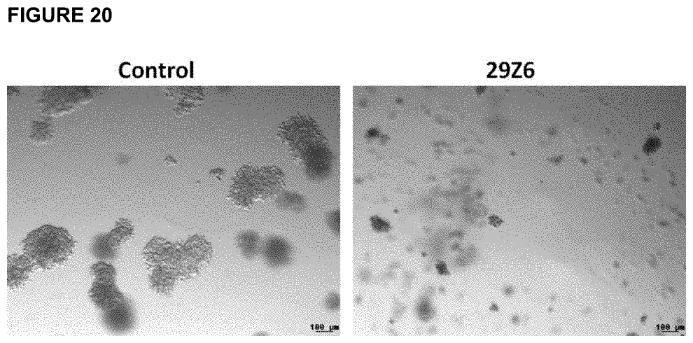

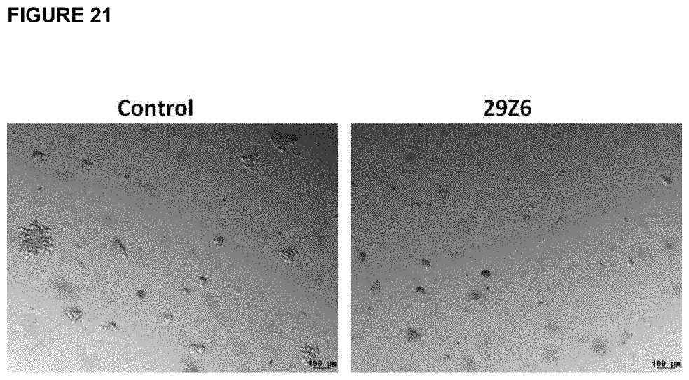

The potential of IgG 29Z6 to inhibit colony formation in various human cancer cell lines (MCF-7, BT-474, MDA-MB-231, PANC-1, FaDu), as marker for cell proliferation, were analyzed. SKBR3 and BT474 were tested because they also express high levels of HER2 and can, thus proliferate in a ligand-independent manner. Cells (1,000 cells per well) were seeded into a 12-well plate in RPMI medium. The next day, cells were incubated with anti-HER3 antibody (IgG 29Z6) at a concentration of 10 μg/ml in RPMI medium containing 2% FCS. After 7 days, medium was removed and fresh medium with antibody at the same concentration was added. At day 19, cells were fixed for 10 min at room temperature and cells were stained with crystal violet for 10 min. Untreated cells (con) were included as negative control. All incubations were performed in quadruplicate. A potent inhibition of colony formation was observed for IgG 29Z6 on all cell lines. 29Z6 antibody supress colony formation in FaDu cells (FIG. 17), 29Z6 antibody supress colony formation in BT-474 cells (FIG. 18), 29Z6 antibody supress colony formation in PANC-1 cells (FIG. 19), 29Z6 antibody supress colony formation in MCF7 cells (FIG. 20), 29Z6 antibody supress colony formation in SK-BR-3 cells (FIG. 21).

### Example 9: In Vivo Studies for 29Z6 Anti-HER3 Monoclonal Antibody

### BxPC-3 Lu Cell Culture:

The pancreatic BxPC-3 Luc tumor cell line will be maintained in vitro as monolayer culture in 1640 medium supplemented with 10% heat inactivated FBS at 37° C. in an atmosphere of 5% CO2 in air. The cells growing in an exponential growth phase will be harvested and counted for tumor inoculation.

### Method for BxPC-3 Lu Tumor Inoculation:

Each mouse will be inoculated subcutaneously on the right flank with the single cell suspension of 95% viable tumor cells (1×10⁷) in 0.1 ml of 1640 medium and Matrigel mixture (1:1 ratio) without serum for the tumor development. Treatment twice a week with 29Z6 antibody (22 mg/kg i.p) or vehicle (control) started when mean tumor size reaches approximately 100 mm³, but not bigger than that. Each group consisted of 10 mice.

### BxPC-3 Lu Tumor Measurements:

The measurement of tumor size will be conducted twice a week with a caliper and the tumor volume (mm³) will be estimated using the formula: TV=a×b²/2 throughout the study, where “a” and “b” is long and short diameters of a tumor, respectively.

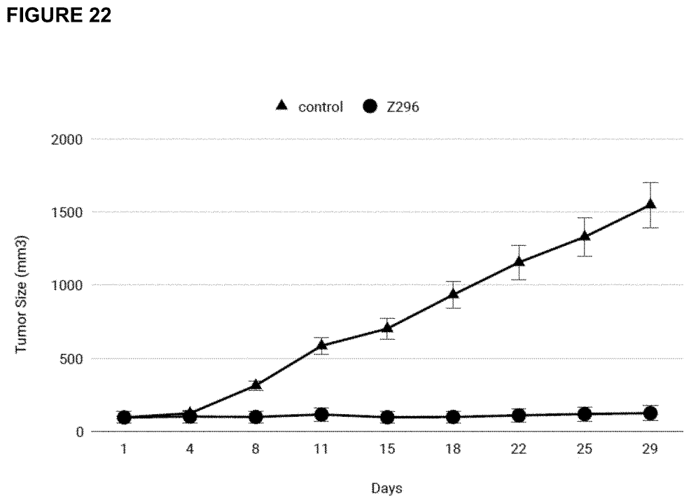

Utilizing a human BxPC-3 Luc pancreatic cancer xenograft model grown subcutaneously in nude mice, 29Z6 demonstrated anti-tumor efficacy with 99% tumor growth inhibition was observed with 22 mg/kg administered twice per week for the duration of the study (FIG. 22).

What is claimed is:

1. An isolated antibody or an antigen-binding fragment thereof, which specifically binds to HER3 comprising a heavy chain variable region comprising SEQ ID NO:1, or an amino acid sequence at least 90% identical thereto, and a light chain variable region comprising SEQ ID NO:2.

2. The antibody or antigen-binding fragment of claim 1, wherein the VH comprises an amino acid sequence at least 80% identical to the amino acid sequence of SEQ ID NO: 1, and wherein the VL comprises an amino acid sequence at least 80% identical to the amino acid sequence of SEQ ID NO: 2.

3. The antibody or antigen-binding fragment of claim 1, wherein the VH comprises an amino acid sequence at least 90% identical to the amino acid sequence of SEQ ID NO: 1, and wherein the VL comprises an amino acid sequence at least 90% identical to the amino acid sequence of SEQ ID NO: 2.

4. The antibody or antigen-binding fragment of claim 1, wherein the VH comprises an amino acid sequence at least 95% identical to the amino acid sequence of SEQ ID NO: 1, and wherein the VL comprises an amino acid sequence at least 95% identical to the amino acid sequence of SEQ ID NO: 2.

5. The antibody or antigen-binding fragment thereof of claim 1, which comprises a VH comprising SEQ ID NO: 1 and a VL comprising SEQ ID NO: 2.

6. The method of claim 1, wherein the antibody comprises a heavy chain variable domain comprising SEQ ID NO: 1.

7. The method of claim 1, wherein the antibody comprises a light chain variable domain sequence comprising SEQ ID NO: 2.

8. A nucleic acid molecule comprising a first nucleotide sequence that encodes a heavy chain variable region (VH), or a second nucleotide sequence that encodes a light chain variable region (VL), or both, of an antibody molecule capable of binding to human HER3, wherein the antibody molecule comprises: (a) a VH comprising a VH CDR1 amino acid sequence of SEQ ID NO: 3; a VH CDR2 amino acid sequence of SEQ ID NO: 4, and a VH CDR3 amino acid sequence of SEQ ID NO: 5; and a VL comprising a VL CDR1 amino acid sequence of SEQ ID NO: 6, a VL CDR2 amino acid sequence of SEQ ID NO: 7, and a VL CDR3 amino acid sequence of SEQ ID NO: 8.

9. An expression vector comprising the nucleic acid molecule of claim 8.

10. An isolated host cell comprising the nucleic acid molecule of claim 8.

11. A method of producing an antibody molecule or fragment thereof, comprising culturing the host cell of claim 8 under conditions suitable for gene expression, wherein said host cell comprises said first nucleotide sequence that encodes a VH and said second nucleotide sequence that encodes a VL.

12. The nucleic acid molecule of claim 8, wherein the first nucleotide sequence encodes a VH comprising the amino acid sequence of SEQ ID NO: 1; and/or wherein the second nucleotide sequence encodes a VL comprising the amino acid sequence of SEQ ID NO: 2.

13. An isolated nucleic acid comprising a sequence encoding the antibody or antigen-binding fragment according to claim 1.

14. An isolated nucleic acid comprising a sequence encoding at least the heavy chain and the light chain of the antibody according to claim 1.

15. The antibody or antigen-binding fragment of claim 1, wherein the antibody is a monoclonal antibody, human antibody, a humanized antibody, a chimeric antibody, a recombinant antibody, a multispecific antibody, or an antigen-binding fragment thereof; wherein the antigen-binding fragment is an Fv, Fab, F(ab′)2, Fab′, dsFv, scFv, or sc(Fv)2; or a diabody, ScFv, SMIP, single chain antibody, affibody, avimer, nanobody or a single domain antibody wherein the antibody or antigen-binding fragment thereof is conjugated to at least one heterologous agent.

16. The antibody or antigen-binding fragment of claim 1, wherein the antibody is a monoclonal antibody.

17. The antibody according to claim 1, wherein the antibody isotype is selected from the group consisting of an IgG1, an IgG2, an IgG3, an IgG4, an IgM, an IgA1, an IgA2, an IgAsec, an IgD, and an IgE antibody.

18. The anti-HER3 antibody of claim 1, wherein the antibody is an IgG2 isotype.

19. The antibody molecule of claim 1, which comprises a light chain constant region of kappa or lambda.

20. A method for treating a subject having a HER3-expressing cancer comprising administering an effective amount of an antibody of claim 1 to the subject.

21. The method of claim 20, wherein the subject is human.

22. The method of claim 20, wherein the subject is a human and the cancer is selected from the group consisting of is a breast cancer, lung cancer, head & neck cancer, prostate cancer, esophageal cancer, tracheal cancer, skin cancer brain cancer, liver cancer, bladder cancer, stomach cancer, pancreatic cancer, ovarian cancer, uterine cancer, cervical cancer, testicular cancer, colon cancer, colorectal cancer or skin cancer. multiple myeloma, gastric cancer, acute myeloid leukemia, chronic myeloid leukemia, osteosarcoma, squamous cell carcinoma, peripheral nerve sheath tumors, renal cancer, malignant mesothelioma, neurofibromatosis benign prostatic hyperplasia, gynacomastica, and endometriosis.

23. The method of claim 20 wherein said tumor is a primary tumor or a metastatic tumor.

24. The method according to claim 20, wherein the lung cancer is non-small cell lung (NSCL) cancer.

25. The method according to claim 20, wherein the cancer of the head or neck is squamous cell carcinoma of the head and neck.

26. The method according to claim 20, wherein the cancer is pancreatic cancer.

27. The method according to claim 20, wherein the cancer is a breast cancer.

28. The antibody of claim 1, wherein said anti-human HER3 antibody or fragment inhibits NRG1-rearranged cancers.

29. The antibody of claim 1, wherein said anti-human HER3 antibody or fragment inhibits cancers with one or more of NRG1-rearranged fusions: (Cluster of Differentiation 74-Neuregulin-1) CD74-NRG1 fusion, (Solute Carrier Family 3 Member 2-Neuregulin-1) SLC3A2-NRG1 fusion, (Syndecan-4-Neuregulin-1) SDC4-NRG1 fusion, DOC4-NRG1 fusion, (Rho-associated protein kinase 1-Neuregulin-1) ROCK1-NRG1 fusion, (Forkhead Box A1-Neuregulin-1) FOXA1-NRG1 fusion, (A-Kinase Anchoring Protein 13-Neuregulin-1) AKAP13-NRG1 fusion, (Thrombospondin 1-Neuregulin-1) THBS1-NRG1 fusion, (Phosphodiesterase 7A-Neuregulin-1) PDE7A-NRG1 fusion, (ATPase Na+/K+ Transporting Subunit Beta 1-Neuregulin-1) ATP1B1-NRG1 fusion, NRG1-PMEPA1 fusion, Clusterin-NRG1 fusion.

30. A patient stratification method where tumors are screened first for NRG1-rearranged fusions and then patients with positive NRG1-rearranged fusions are treated with anti-human Her3 antibody of claim 1.

31. The method of claim 29, further comprising, prior to the administering, using a method that comprises analysis of a predictive marker to select a subject having a disease associated with HER3.

32. The method of claim 29, further comprising an additional therapeutic agent.

33. The method of claim 29, wherein the additional therapeutic agent is selected from the group consisting of an EGFR inhibitor, a HER2 inhibitor, a HER3 inhibitor, a HER4 inhibitor, an mTOR inhibitor and a PI3 Kinase inhibitor.

34. The method of claim 29, wherein the additional therapeutic agent is a EGFR inhibitor selected from the group consisting of Matuzumab (EMD72000), Cetuximab, Panitumumab, mAb 806, Nimotuzumab, Gefitinib, CI-1033 (PD183805), Lapatinib (GW-572016), Lapatinib Ditosylate, Erlotinib HCL (OSI-774), PKI-166, and N-[4-[(3-Chloro-4-fluorophenyl)amino]-7-[[(3″S″)-tetrahydro-3-furanyl]o-xy]-6-quinazolinyl]-4(dimethylamino)-2-butenamidea HER2 inhibitor selected from the group consisting of Pertuzumab, Trastuzumab, MM-111, neratinib, lapatinib or lapatinib ditosylate/Tykerb®; a HER3 inhibitor selected from the group consisting of, MM-121, MM-111, IB4C3, 2DID12 (U3 Pharma AG), AMG888 (Amgen), AV-203 (Aveo), MEHD7945A (Genentech), MOR10703 (Novartis) and small molecules that inhibit HER3; and a HER4 inhibitor.

35. The method of claim 29, wherein the additional therapeutic agent is an mTOR inhibitor selected from the group consisting of Temsirolimus, ridaforolimus/Deforolimus, AP23573, MK8669, and everolimus.

36. The method of claim 29, wherein the additional therapeutic agent is a PI3 Kinase inhibitor selected from the group consisting of GDC 0941, BEZ235, BMK120 and BYL719.

37. The antibody of claim 29, wherein the antibody is conjugated to an imaging agent, therapeutic or a chemotherapeutic agent, a toxin or a radionuclide.

38. The method of claim 29, wherein said therapeutic or chemotherapeutic group is selected from the group consisting of calicheamicin, auristatin-PE, geldanamycin, maytansine and derivatives thereof.

39. The method of claim 1, wherein the antibody or fragment thereof is administered by a route selected from the group consisting of oral, subcutaneous, intravenous injection intraperitoneal, intramuscular, intracerebroventricular, intraparenchymal, intrathecal, intracranial, buccal, mucosal, nasal, and rectal administration.

40. The method of claim 1, wherein the antibody or fragment is formulated into a pharmaceutical composition comprising a physiologically acceptable carrier, excipient, or diluent.
